(19) United States
(12) Patent Application Publication
(10) Pub. No.: US 2021/0035400 A1
(43) Pub. Date: Feb. 4, 2021

(54) System, Apparatus, and Method for the Automated Purchasing and Dispensing of Medications

(71) Applicant: Wellmation, LLC, Lakeland, FL (US)

(72) Inventors: Ryan Flynn, Tampa, FL (US); Zachary Johnson, Wesley Chapel, FL (US); Roger H. Grow, Lafayette, CO (US)

(21) Appl. No.: 16/944,780

(22) Filed: Jul. 31, 2020

Related U.S. Application Data
(60) Provisional application No. 62/971,409, filed on Feb. 7, 2020.
(60) Provisional application No. 62/881,478, filed on Aug. 1, 2019.

(51) Int. Cl. G07F 11/54; G16H 40/67; G06Q 40/08; G16H 20/13

(52) U.S. Cl. CPC G07F 11/54; G16H 20/13; G06Q 40/08; G16H 40/67

(57) ABSTRACT

Disclosed is an apparatus for securely storing, labeling, and automatically dispensing a medication to a patient. The apparatus includes an interface for verifying the identity of the patient prior to the medicine being dispensed. The patient obtains the medication via a dispenser that is selectively rotatable between secure and dispensing sides. The disclosure further relates to an associated system and method for allowing a health care professional to remotely prescribe a medication that is subsequently dispensed by the apparatus.

### CROSS REFERENCE TO RELATED APPLICATIONS

This application claims priority to and is a continuation of co-pending Provisional Patent Application Ser. No. 62/881,478 filed on Aug. 1, 2019 and entitled “System, Apparatus, and Method for Remotely Prescribing and Automatically Dispensing Medications.” This application also claims priority to and is a continuation of co-pending Provisional Patent Application Ser. No. 62/971,409 filed on Feb. 7, 2020 and entitled “System, Apparatus, and Method for the Automated Purchasing and Dispensing of Cannabis.” The contents of both these applications is incorporated herein for all purposes.

### FIELD OF THE INVENTION

This disclosure relates to a system, apparatus, and method for the automated purchasing and dispensing of medications. The disclosure further relates to an automated cannabis dispensing system with associated security and authentication protocols.

### BACKGROUND OF THE INVENTION

The traditional method for dispensing medications requires an authorized individual, such as a medical technician, pharmacist or druggist, to be physically present at a predetermined location where medications are stored. Upon receiving appropriate documentation from an individual with a prescription, the authorized individual has the medication manually counted, verified, and then dispensed in accordance with the specific prescription or needs of the individual patient. The terms medicine or medication as used herein broadly encompasses any substance that is used for medical treatment. This includes medications that can only be dispensed on the order of a physician, physician assistant, or qualified nurse, as may be required for controlled substances and scheduled drugs. The term further includes various over-the-counter medications. Some of these over-the-counter medications may require a prescription or may be subject to use or sale restrictions. Other over-the-counter medications will not require any prescription and will not be subject to any use or sales restrictions. All of the foregoing are encompassed by the terms medicine or medication as used herein.

For medications, and particularly prescription medication, a health care professional typically examines a patient, reviews their medical history, and determines the patient's physical condition (either in person, or remotely through tele-medicine). The health care professional then prescribes a particular medication based on a patient's condition or need, often by use of a hand written prescription. In these instances, a patient takes the written prescription to a pharmacist at a pharmacy location for fulfillment of the prescription. In such manual in-person dispensing, the pharmacist verifies the identity and authorization of the patient to receive the medication through personal identification, determines the payment distribution based on the patient's insurance card, and contacts the prescribing doctor for verification if needed.

Some advancements have been introduced into the process of dispensing medication and some steps have been simplified through transmission using electronic means. In the case of prescription drugs, prescriptions are often transmitted from a doctor to a pharmacy via an online portal or other software-based system. Nonetheless, a patient or user of a medication, particularly when the medication is a controlled substance, is still required to physically travel to the location of the medication, where the authorized dispensing personnel is located, to retrieve the medication.

Some attempts have been made to use automatic prescription drug dispensers to dispense drugs at various locations. These dispensers have numerous limitations, including the inability to support foreign languages. These dispensers have also been reported as being unfriendly to users. The results of studies conducted on the viability of these dispensers shows that in most hospital settings, the assistance of staff and employees has been required to assist individuals utilizing the devices.

There remains a need for providing an integrated and automated kiosk device connected to a comprehensive system that provides security, authentication, and protections for controlling access to medications by personnel other than authorized handlers of the medications. Further, there remains a need for a device that communicates with an overall system utilized by the healthcare provider, or pharmacy provider, and is safely accessed by individual patients and users of medications. There remains an unmet need to provide for security and controlled access to medications at locations remote from pharmacies and authorized dispensing personnel, and that does not require pharmacist review or intervention. Upon authentication and authorization of an individual patient/user, what is needed is a fully automated process that occurs internally within the kiosk system controlled by a robotic system, without need for confirmation or assistance by a human.

Additionally, there remains an unmet need for a system and apparatus that a patient, or user of a medicine, can access during hours that a pharmacy or other location would typically be closed. Standard working hours and personal and family commitments often conflict with normal pharmacy hours making it difficult to pick-up prescriptions during the hours that pharmacies are open. A system, apparatus and method that allows for verification of identity and prescription information in advance, and that allows the user/patient to pick-up a pre-ordered/pre-filled prescription at will, and that eliminates the need for a human to be present when medications are dispensed, is desired.

There further remains a need for a device that can be accessed twenty-four hours a day, seven days a week, including holidays, where a patient, or user of a controlled substance, can pick up a prescription, at a time that works for their schedule. Illness and sickness are often unpredictable. For example, when an individual becomes sick and visits their doctor's office the day before a holiday, they are often unable to access the medicines that they are prescribed because the pharmacy, or other medical type dispensary is closed for the holiday by the time the prescription can be filled. What is needed is an apparatus that can pre-authorize and authenticate an individual, process insurance claims and payment, and can fill these types of emergency prescriptions at any hour, and on any date, including holidays. Thus there remains an unmet need for a user-friendly kiosk that is available at all hours, that provides a friendly, simple and straight forward user interface system, that also functions as a safe and automated medicine dispensary. The system, apparatus, and method of the present disclosure are aimed at overcoming these as well as other shortcomings present in the background art.

The present disclosure also relates to a system, method and apparatus for the automated dispensing of cannabis. Cannabis use is increasing across the United States and around the world as more governments legalize medical and recreational uses. Currently eleven states and the District of Columbia have legalized the recreational use of cannabis. Furthermore, 33 states and the District of Columbia have legalized the medical use of cannabis. These numbers will only increase over time. The U.S. cannabis industry is worth billions of dollars and all signs point to ever increasing sales. Despite increasing acceptance, cannabis sales are often subject to strict government regulations. These regulations can include, for example, age restrictions, sales limits, and packing and labeling requirements. Additional regulations, either in place or anticipated, address the need to authenticate purchasers, verify prescriptions, and track daily or weekly limits on purchases. Still yet other regulations require sellers to keep accurate inventories for the purpose of documenting all transactions from “seed to sale.” Tracking these regulations becomes more complex when considering that they vary from state to state and even from city to city. The burgeoning nature of the industry also requires cannabis sellers to anticipate and be ready to comply with future regulations. However, current dispensing systems do not have adequate security protocols to comply with current or anticipated future regulations.

What is needed, therefore, is an automated system for dispensing cannabis that includes appropriate safety and security protocols. What is also needed in the art is a dispensing system that can be readily configured to comply with known and anticipated future regulations regarding the sale and distribution of cannabis. There is a further need in the art for an automated dispenser that can comply with both regulations governing recreational and medical cannabis. The automated system of the present disclosure fulfills these and other needs present in the art.

### SUMMARY OF INVENTION

The present invention provides a system apparatus and method for the automated dispensing of one or more medications to an authorized and authenticated patient/user. The system allows a medication, prescribed by a health care provider (such as a physician, physician assist, or qualified nurse) to be automatically dispensed at a remote location to an authenticated and verified user, upon command. Particularly where the medication is a controlled substance, a health care provider (such as a doctor, nurse practitioner, or physician's assistant) uploads a prescription order for a patient, using prescription ordering software that can communicate with the overall kiosk system software and kiosk dispensing device. The system further includes a central processing system which communicates with the ordering software to receive one or more orders for medications for an identified and verified patient, and one or more kiosks at remote locations to verify the availability of fulfilling the order.

When a user/patient approaches the device and interacts with the user interface they are prompted to confirm their identity by inserting their driver license, ID card or other identification means. The device scans and extracts the information/data contained on the user/patient's identification card and transmits the information/data through built-in software for verification, possibly via an authentication agent. A camera, or other imaging device, may be built into the kiosk to scan an image of the user/patient and confirm their identity. Other methods of verifying a patient's identity can also be employed, such as biometric identification.

Upon confirmation of the user/patient's identity, a method for dispensing the controlled substance to the user/patient is automatically initiated. A robot system housed inside of the pharmacy vault where the inventory of medications is stored, in a fully automatic process, selects the preauthorized medication that matches the prescription ordered by the patient/user, and labels the containers with the patient/user's specific identification information, instructions for use, dosage, and other information appropriate and commonly included on prescription medication bottles. Transaction information, alone or with prescription information, can be included on the label. Nothing herein is intended to limit what may be contained on such labels. Following the labeling of the container, the robot systems moves the prescription container into the shelving slots of the revolving container dispensing portion. The labeling of the container can also be carried out prior to the patient's arrival. The revolving container dispensing portion, rotates from the interior pharmacy vault of the kiosk, to the exterior of the kiosk, such that the user/patient can then access and pick-up their prescription. The use of the revolving container dispensing portion, prevents unauthorized access to the interior of the pharmacy vault and other controlled substances.

Following the dispensing of a medication, the built-in software updates the kiosk inventory on the central processing system software. The automatic inventory update is sent in real-time, from the kiosk to the central processing system, in order to avoid a user having to travel to a specific kiosk, and then find out that a specific medication is unavailable. When inventory volume reaches certain preset levels, the central processing system will send a command to an authorized entity or individual to refill a specified medicine, in order to decrease or prevent the unavailability of the medication.

When the kiosk needs refilling, the container loading portion of the kiosk may be refilled by an authorized representative of the company responsible for refilling medications. The container loading portion is comprised of a rotatable door. One side of the container loading portion is comprised of an exterior surface that serves as the exterior wall of the container loading portion when the kiosk is not being refilled. The opposite side of the container loading portion is comprised of a shelving portion, where the controlled substances can be placed. Once the shelving is filled by the responsible company, it rotates back inside to the interior of the pharmacy vault, where the robotic arm system will move and store the controlled substances accordingly, within the container holding portion. This controlled rotatable container loading portion, prevents unauthorized access to the interior of the pharmacy vault. A similar rotatable container dispensing portion can also be employed to allow a patient to retrieve their medication.

The interior of the kiosk is comprised of at least one container holding portion, where numerous medications can be stored and organized, in a safe and controlled environment, accessible and controlled solely by the robot system. This area may be accessed by certain authorized personnel when necessary, but generally will not be accessible. This apparatus and device is temperature and humidity controlled to ensure the integrity of the medicines stored inside. It should be appreciated that the device and apparatus may contain various medicines including all different schedules of medicines, creams, and antibiotics. It is also within the scope of the present disclosure to employ the kiosk in the dispensing of prescribed products, such as contact lenses or orthotics.

The kiosk is comprised of two main vaults, a pharmacy vault and a system vault. The electrical control panel of the user interface, the controlled substance labeling machine, and other electrical components of the kiosk are all stored in a separate system vault of the device, such that the pharmacy vault where the controlled substances are located, should never need to be accessed by general maintenance or repair persons. Individuals performing maintenance on the machine, power supply, computer system or refilling the label maker can access the system vault in which these components are stored without gaining access to the medicines stored in the other pharmacy vault.

It is therefore an object of the present disclosure to provide a fully automated system that allows for the selection, purchase, and dispensing of items such as medications or cannabis.

It is another object of this disclosure to provide a single kiosk dispenser that can hold a wide variety of medications and that can alternatively be used for medically prescribed or recreational cannabis.

Yet another object of this disclosure is to provide an automated kiosk that complies with known governmental regulations but without the need for a human to monitor or control the transaction.

A further object of this disclosure is to provide a kiosk that can be easily modified to comply with future, anticipated regulations regarding the sale of medications or cannabis.

The kiosk of the present disclosure is comprised of two main vaults, a first container vault and a second system vault. The electrical control panel of the user interface, the container labeling machine, and other electrical components of the kiosk are all stored in the second system vault of the device, such that the first container vault where the substances are located, should never need to be accessed by general maintenance or repair persons. Individuals performing maintenance on the machine, power supply, computer system or refilling the label maker can access the second system vault in which these components are stored without gaining access to the products stored in the other container vault.

### DESCRIPTION OF FIGURES

The foregoing and other objects, features and advantages of the invention will be apparent from the following more particular description of preferred embodiments of the invention, as illustrated in the accompanying drawings in which like reference characters refer to the same parts throughout the different views. The drawings are not necessarily to scale, emphasis instead being placed upon illustrating the principles of the invention.

FIG. 1 shows the device with an individual user/patient standing in front of the user interface panel.

FIG. 2 shows one embodiment of the device, showing the first pharmacy vault that stores the medications, the second system vault that holds the electrical components and user interface, and a third vault that houses methods for reading the user's identification card.

FIG. 3 shows the device with the first door to the pharmacy vault open and the door to the system vault open.

FIG. 4 shows the device with the second door to the pharmacy vault open and the door to the system vault open.

FIG. 5 shows the device with the door to the system vault that holds the electrical components to the device open.

FIG. 6 shows the interior of the vault that holds the electrical components to the device, specifically the power supply, container labeling machine and the discarded prescription box.

FIG. 7 shows the container labeling machine, pulled down from its storage position, to allow for refilling of labels.

FIG. 8 shows the device with the first door of the pharmacy vault open.

FIG. 9 shows the device with the first and second door of the pharmacy vault that holds the medications open, showing the interior of the vault, the shelving portions, and the robot system.

FIG. 10 shows the interior shelving portion of the second door of the pharmacy vault that holds the medications, including the shelving portion of the container storage portion, the shelving portion of the container dispensing portion, and the shelving portion of the container loading portion.

FIG. 11 shows a container stored on the shelving portion.

FIG. 12 shows the robot system holding a container.

FIG. 13 shows how the robot system moves and rotates, removing a container from the container storage portion and moving it to the container labeling portion.

FIG. 14 shows an embodiment of the kiosk with shelving on the back side of the system vault.

FIG. 15 shows the container dispensing portion midway in its rotation from the interior of the pharmacy vault, outwards, to the exterior of the pharmacy vault to dispense a medicine to a user/patient.

FIG. 16 shows the container dispensing portion facing the exterior of the pharmacy vault, dispensing a medicine to a user/patient.

FIG. 17 shows the container loading portion midway in its rotation from the interior of the pharmacy vault, outwards, to the exterior of the pharmacy vault to be refilled with medicine.

FIG. 18 shows the container loading portion of the device being refilled with medicine containers.

FIG. 19 shows an embodiment of the device where the back panel has a slot leading into a container station where discarded controlled substances can be stored, until they can be picked up and removed.

FIG. 20 shows a detailed schematic of the overall system that details the interaction between the prescription management/inventory software system, doctor's offices, kiosks, controlled substance handlers and users/patients.

FIG. 21 illustrates two labeled containers.

FIG. 22 shows the dispensing and loading portions of the kiosk.

FIG. 23 is a plan view of the dispensing portion taken along line 23-23 of FIG. 22.

FIG. 24 is a plan view of the loading portion taken along line 24-24 of FIG. 22.

FIG. 25 is a detailed view of the robotic gripper.

FIG. 26 is a further detailed view of the robotic gripper.

FIG. 27 is an additional detailed view of the robotic gripper.

FIG. 28 is a plan view of a store room facility and kiosk vault.

Similar reference numerals reference similar elements throughout the several views of the drawings.

### PARTS LIST

| | |
|---|---|
| 50 Master Kiosk Vault | 200 System Vault |
| | 201 System access door |
| 100 Pharmacy Vault | 202 User Interface |
| 101 Pharmacy access door | 203 Camera |
| 102 Television Screen | |
| 104 Locking Mechanism | 210 Removable Panel |
| | 211 Keypad |
| 110 Prescription dispensing portion | 212 Credit card reader |
| 111 Exterior side of door | 213 Receipt dispenser |
| 112 Interior side of door | 214 Phone |
| 120 Container loading portion | 220 Power Supply |
| 121 Exterior side of door | 221 Rear wall |
| 122 Interior side of door | 222 External side wall |
| | 223 Internal side wall |
| 130 Pharmacy vault door | 224 Computers and hardware |
| 131 Rear wall | 225 Cooling and heating elements |
| 132 External side wall | 226 Receipt paper spool |
| 133 Internal side wall | 227 Discarded container station |
| 134a “V-shaped” shelves (vault door) | 228 Locking mechanism |
| 134b “V-shaped” shelves (vault wall) | 229 Chute to container station |
| 134c “V-Shaped” shelves (back of | 230 Label generating device |
| System Vault) | |
| 134d “V-shaped” shelves (loading) | 231 Labels |
| 134e “V-shaped” shelves | 232 Spool for backs of labels |
| (dispensing) | |
| 136 Container labeling station | 233 Adjustable shelf |
| 137 Container storing portion | 234 Spool for blank labels |
| | 235 Printer with motor |
| 140 Robotic system | |
| 141 Bottle gripper | 300 Third Vault |
| 142 Motor | 301 Card Slot for Identification |
| 143 Scanner | 302 Cash Payment Processing |
| | Device |
| 144 Vertical axis | |
| 145 Horizontal track | 400 System |
| 146 Picking arm | 410 Remote Computing Terminal |
| 147 Proximity Sensor | 412 Health care facility |
| 148 Camera | 422 Centralized Processing System |
| | 430 Kiosk |
| 150 Containers | 432 Pick-up location |
| 151 Fingers | |
| 152 Housing | 502 Store Room |
| 153 Biasing Spring | 504 Secure Door to Store Room |
| 154 Central Support | |

### DETAILED DESCRIPTION

The present disclosure relates to an apparatus for securely storing, labeling, and automatically dispensing a medication to a patient. The apparatus can be used to dispense any of a variety of medications. The apparatus includes an interface for verifying the identity of the patient prior to the medication being dispensed. In one embodiment, the apparatus can be stocked with medications introduced into a pharmacy vault through a rotatable door and subsequently fulfilled by an automated robot within the pharmacy vault. A rotatable dispensing door is then used to deliver the medication to the patient. The disclosure further relates to systems and methods for allowing health care workers to remotely prescribe a medication, authenticate the patient's ability to receive it, and process associated insurance information and payment. The various features of the present disclosure, and the manner in which they interrelate, are described in greater detail hereinafter.

### Automated Dispensing Apparatus

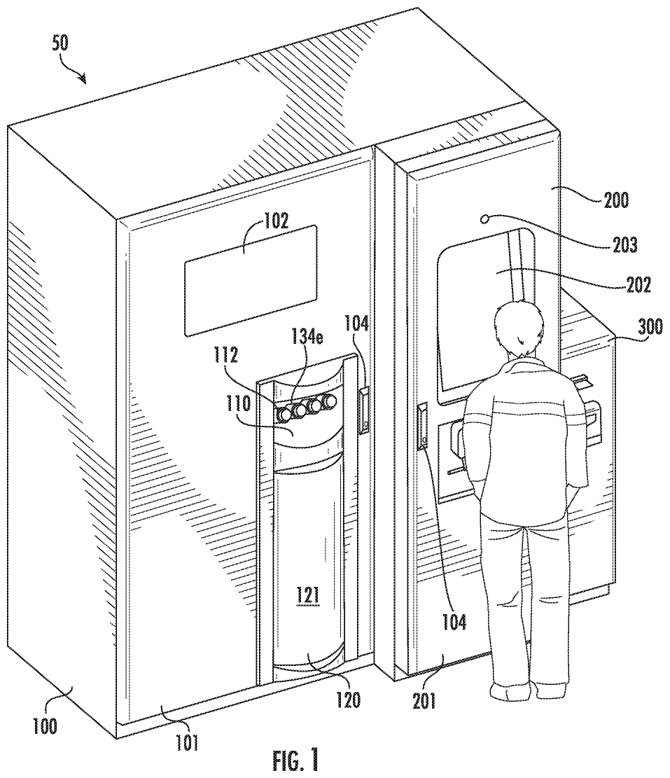

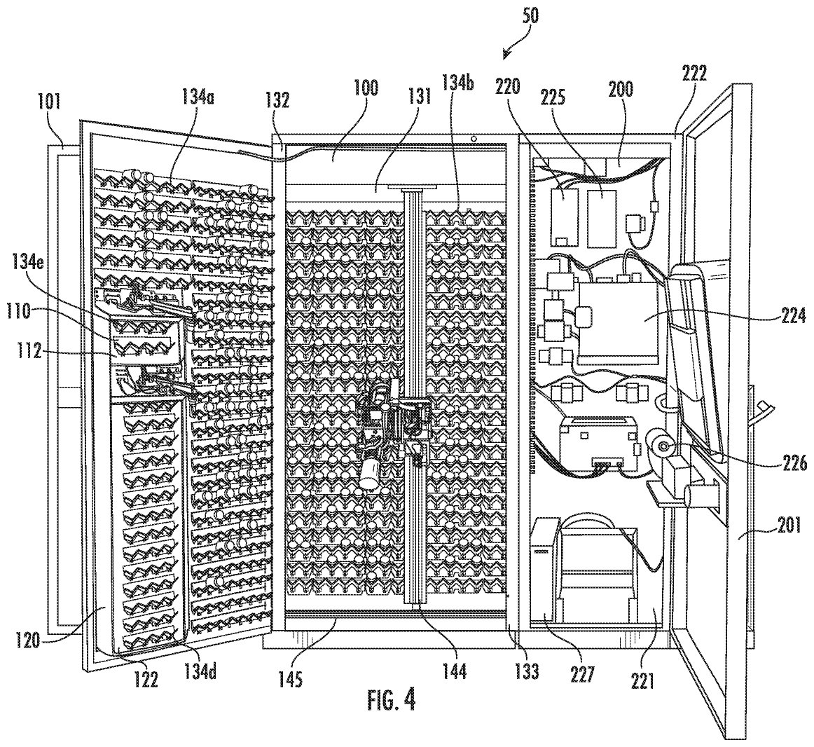

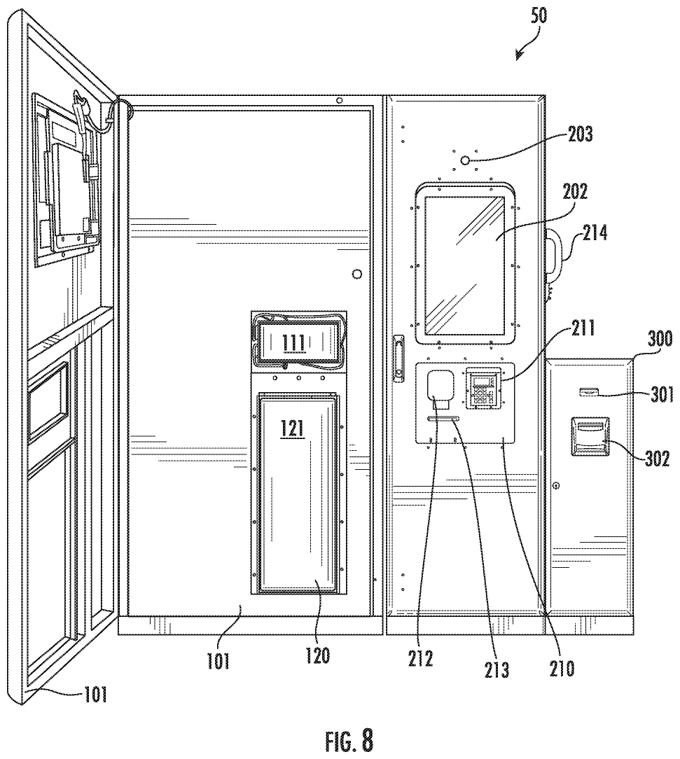

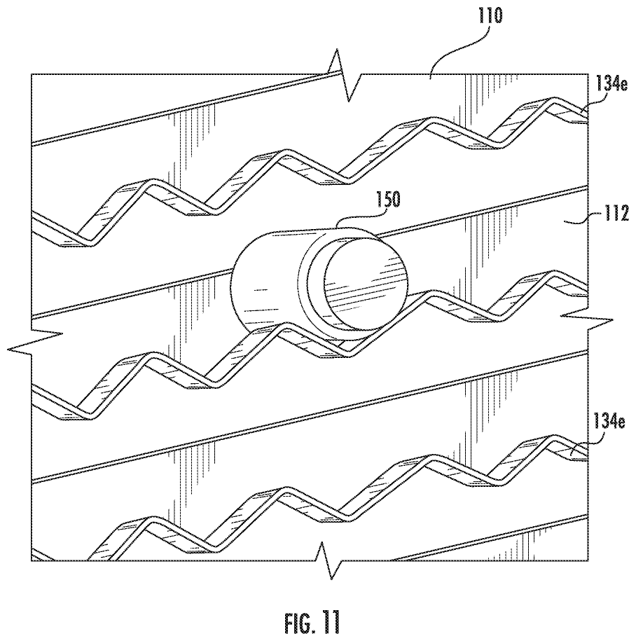

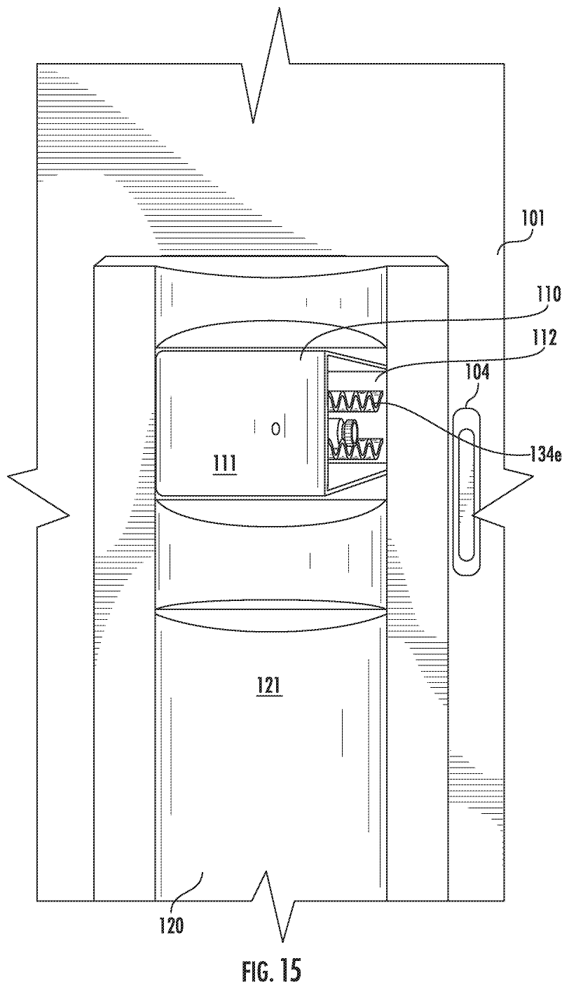

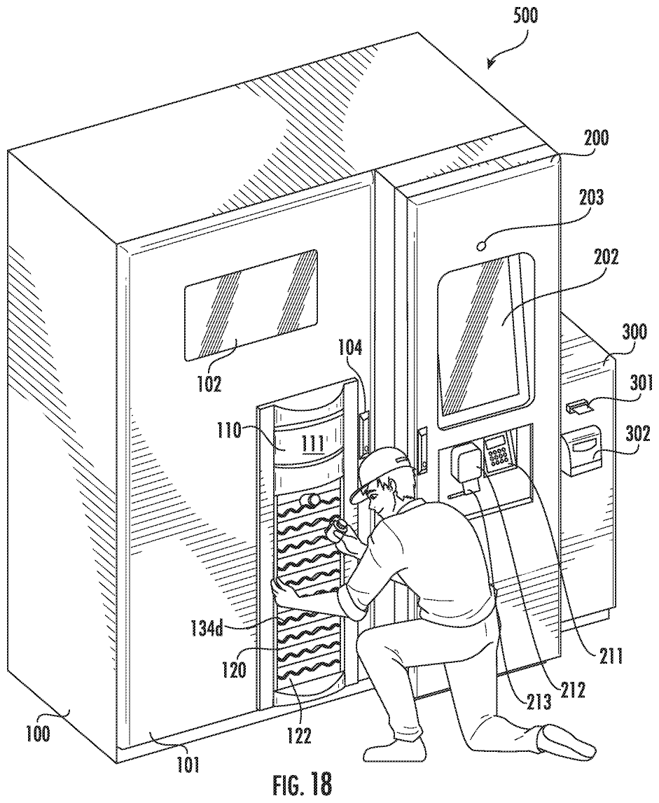

Master kiosk vault (50) (illustrated in FIGS. 1-19) is employed in storing, labeling, and automatically dispensing containers (150). Containers (150) may store any of a wide variety of medications. The term medication as used herein broadly encompasses any substance that is used for medical treatment. This includes medications that can only be dispensed on the order of a physician, physician assist, or qualified nurse, as may be required in the U.S. under the Controlled Substances Act or other applicable regulations. The term further includes various over-the-counter drugs, some of which may nonetheless require a prescription or that may be subject to use or sale restrictions. As noted below, containers (150) may alternatively contain cannabis. Kiosk (50) allows containers (150) to be securely loaded, stored, automatically labeled, and then, upon the identity of the patient being verified, dispensed. All of this is achieved via a secure kiosk (50) that limits access to authorized individuals.

### Master Vault

As shown in FIG. 1, kiosk (50), or master vault, includes both a pharmacy vault (100) and a system vault (200), each with an interior area. As described below, pharmacy vault (100) securely houses medicine containers (150), while system vault stores associated electronics and communications systems. Kiosk (50) includes access panels in the form of two doors, a pharmacy access door (101), and a system access door (201). Pharmacy vault (100) further includes a second, interior vault door (130) that controls access to the medicine containers (150). Each of the doors (101, 201, and 130) is hingedly secured to its associated vault (100 and 200) and each includes opened and closed positions. Interior vault door (130) is optionally more secure than the outer access door (101), and once locked, cannot be accessed without a special key, code, or other authentication means. This limits access to only those individuals authorized to handle the medications stored within the pharmacy vault (100) and prevents access by unauthorized individuals or personnel who may be attending to other aspects of kiosk (50). Authentication means for interior vault door (130) may include codes, keys, cards, digital or electronic authentication means or other authentication means.

Pharmacy access door (101) preferably includes openings for providing access to a dispensing portion (110) and a loading portion (120), both of which are mounted within the vault door (130). The operation and purpose of these portions is described in further detail below. A television screen (102) for use in advertising, or other related purposes, can optionally be installed within access door (101). The pharmacy access door (101) and system access doors (201), further include one or more locking mechanisms (104) to secure the interior of kiosk (50). In some embodiments the locking mechanism (104) may require a key to gain access to the interior of the vaults. In other embodiments, authorized individuals may enter an electronic code, scan a badge, or use other means to open the vault.

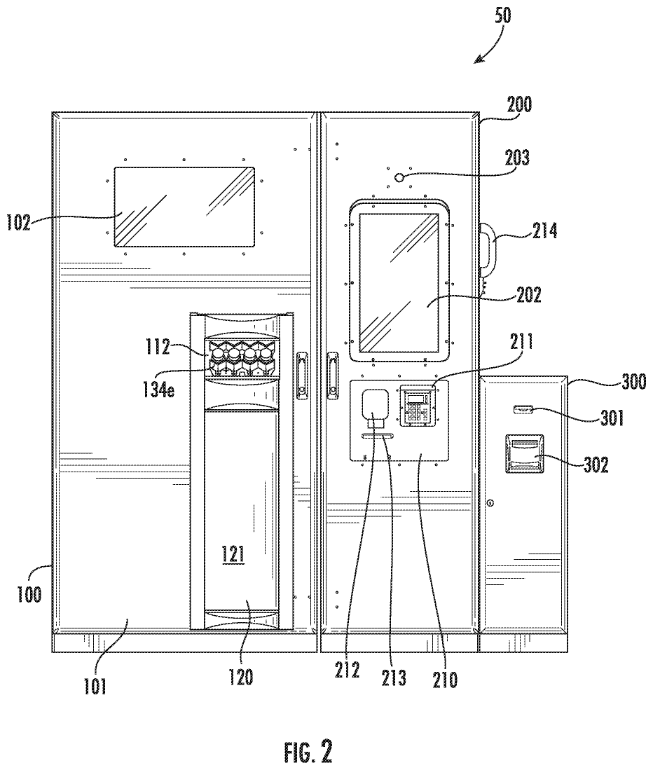

As illustrated in FIG. 2, kiosk (50) optionally includes a third vault (300) for use in processing cash payments and verifying a patient's identity. The patient's identity may be verified by scanning relevant credentials (e.g. a driver's license), scanning or inputting an appropriate code, or via biometrics, such as retina scans, body scans, or voice identification. This third vault (300) may include, for example, a card slot (301) for accepting identification that is compliant with government identification standards (e.g. a driver's license) as well as a cash payment processing device (302). Card slot (301) can preferably read both sides of any government issued identification card.

The system access door (201) is positioned in communication with a user interface (202) and a camera (203), both of which can be used for authenticating patients. For example, interface (202) and camera (203) can be used to conduct a video chat between the patient and an authorized individual who may visually confirm the identity of the patient or who may visually confirm a form of identification presented by the patient. Alternatively, the health care professional writing a prescription for a patient may provide the patient with a pre-defined security code. The patient, upon arriving at kiosk (50), would enter this code via interface (202) (or via a keypad) to confirm their identity. Other methods of authentication can also be used, for example, the user can identify themselves by entering a date of birth, personal identification number, or a member I.D. Any of these verification methods may be carried out before any medication is dispensed to the user. Patient verification can also be carried out via biometrics, such as retina scans, facial recognition, body scans, or voice identification. In the event facial recognition is employed, it can be utilized to confirm that the patient's face matches the photo within the patient's identification. This would avoid the necessity of the patient needing to pre-register with the system.

User interface (202) and camera (203) can also be employed for telepharmacy. Namely, the patient may converse with a remote pharmacist for the purpose of consultation. This may include answering questions about a particular medication or questions about fulfilling the prescription. Interface (202) and camera (203) can likewise be used so that the remote pharmacist can visually verify that the correct medication is being dispensed to an identified patient. For example, the pharmacist can access images taken of the medicine container both before and after the application of a prescription label to verify that the correct medicine is being dispensed and that it has been labeled correctly.

As shown in FIG. 2, the system access door (201) also includes a panel (210) that has a keypad (211), credit card reader (212) and receipt dispenser (213). Credit card reader (212) can be touchless or a dip type reader. Each of these can be used to process credit card payments and provide a receipt to the patient. Panel (210) is preferably removable to allow for convenient and simple interchangeability of replacement panels containing new credit card readers, as technology and requirements for credit card and payment processing systems change and advance.

### System Vault

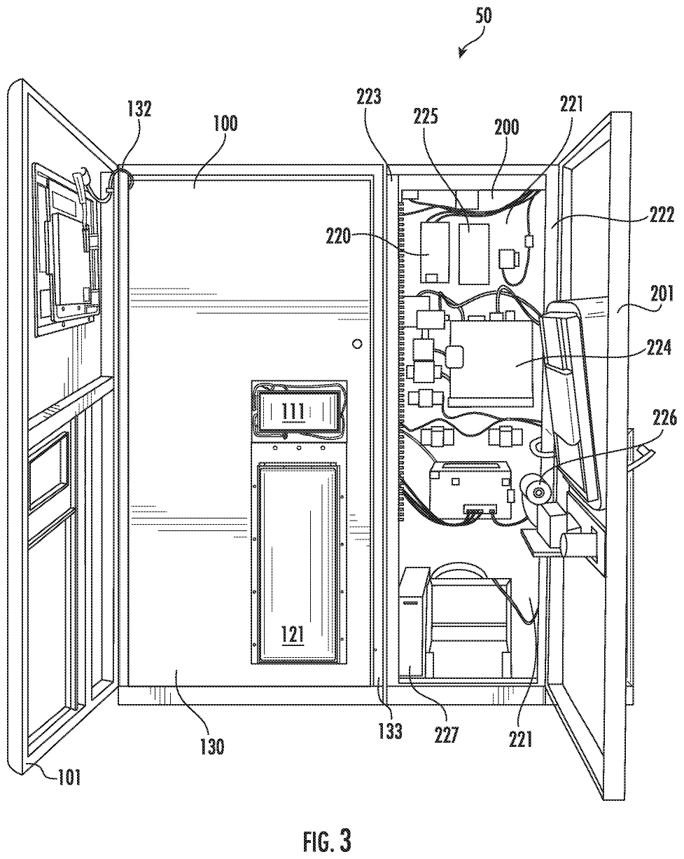

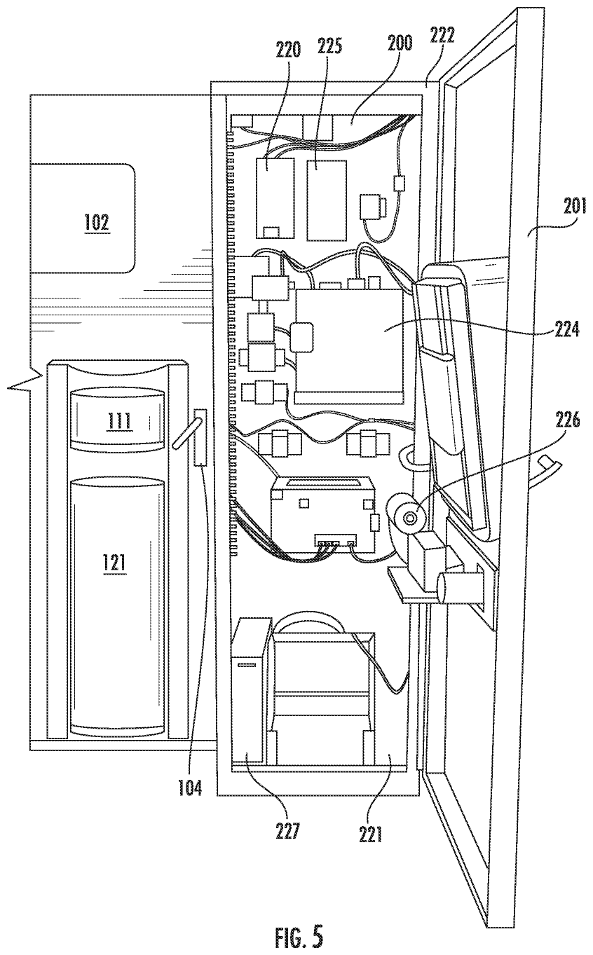

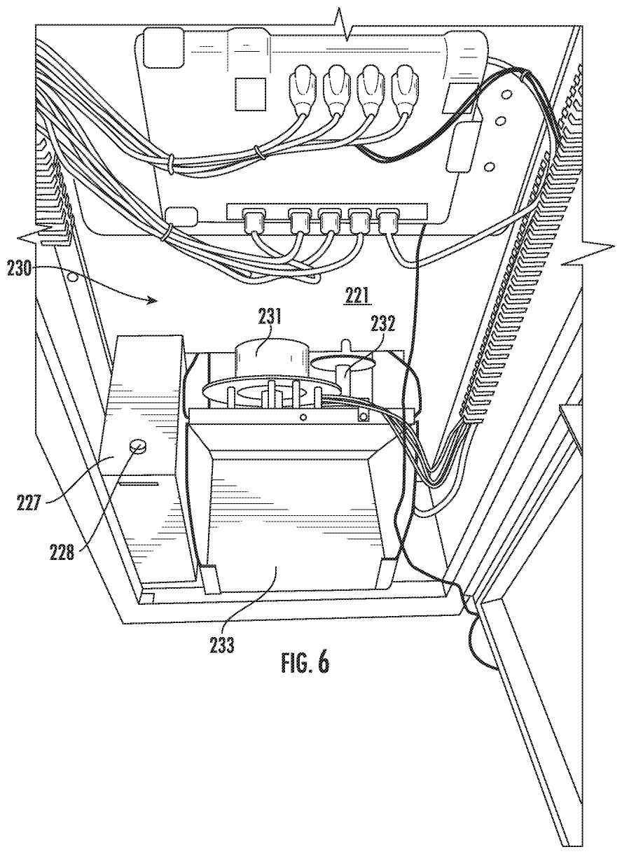

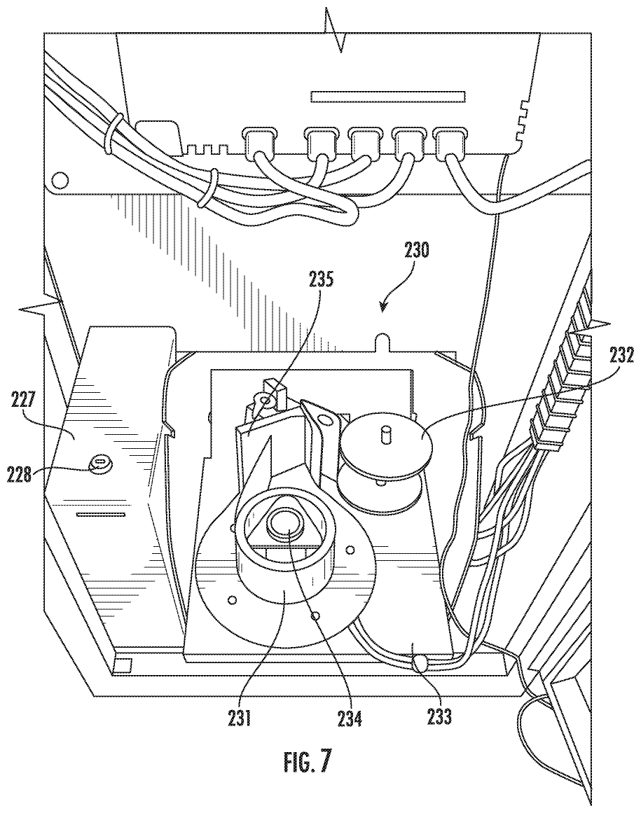

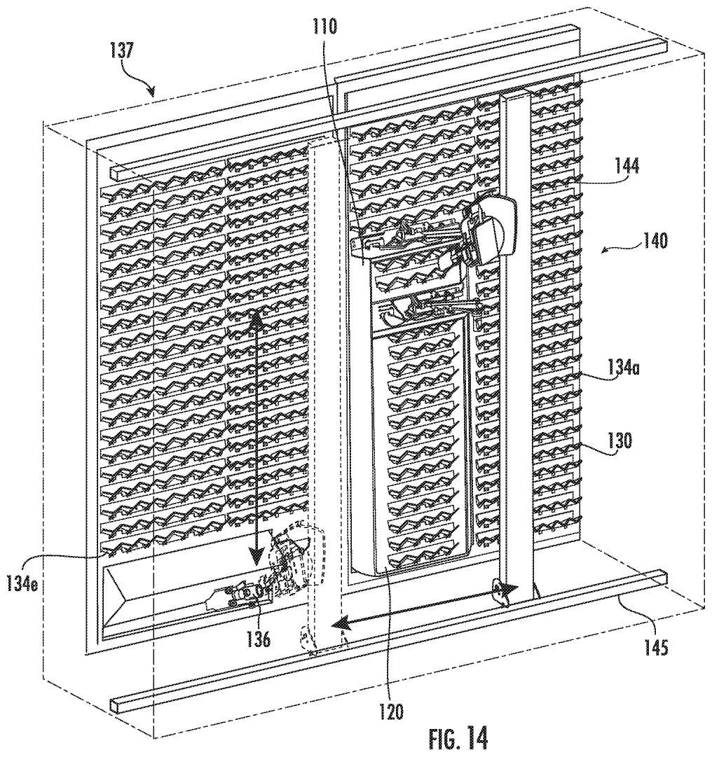

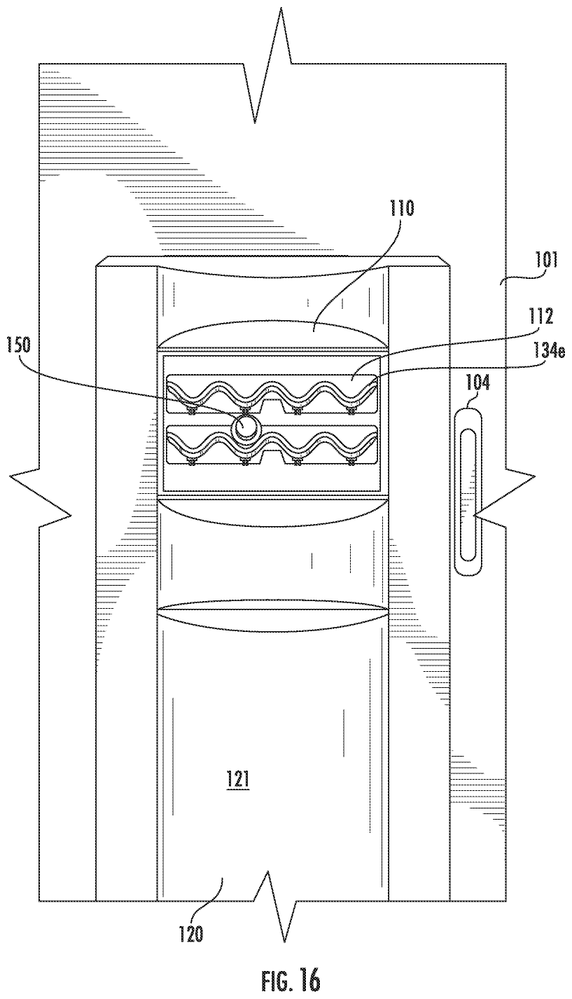

Opening the system access door (201) reveals the interior of the system vault (200) (FIG. 3). System vault (200) is defined by a rear wall (221), an external side wall (222), and an internal side wall (223). As shown in FIG. 14, a plurality of v-shaped shelves (134) may be secured to be back side of system vault (200). Each row of v-shaped shelving may include a protruberance for positively securing containers located on the lower and adjacent row of v-shaped shelving (FIG. 16). Internal side wall (223) is a common wall with the internal side wall of the pharmacy vault (100). As shown in FIG. 5, FIG. 6 and FIG. 7, the system vault (200) contains the power supplies (220), computers and hardware (224), and cooling and heating elements (225), and communications equipment (236) for the kiosk device. A spool (226) of receipt paper may also be included. Computer hardware with system vault (200) may include a central processing unit (224) (“CPU”). CPU (224) preferably runs software systems for communicating (either via a wired or wireless connection) with a central processing system (described below) and for receiving and confirming patient prescriptions and information. CPU (224) may also run software systems for controlling the dispensing machinery (described below).

The system vault (200) further includes a container discard station (227) that is also accessible through the rear wall (221) of the system vault (200). Discard station (227) preferably includes a locking mechanism (228). Discard station (227) allows unwanted, expired, or recalled medicines to be disposed of in a secure manner.

### Label Generator

As illustrated in FIG. 7, system vault (200) also includes a label generating device (230) within its interior area. Label generating device (230) includes a supply of labels (231), a printer (235), motor (234) and a take up wheel (232). These components are preferably all housed on an adjustable shelf (233), which may be pivoted outwardly toward the user to access the various components of device (230) and thereafter pivoted to a closed position for subsequent use. Label generating device (230) is controlled by CPU (224) and is adapted to generate labels containing information relevant to the prescription medication being dispensed. A labeling station (136) is also included within vault (200). Labeling station (136) is ideally accessible through the rear wall (221) of system vault (200). This allows a robotic system (described below) to position the container (150) into station (136) for labeling, without the need for removing the container (150) from pharmacy vault (100).

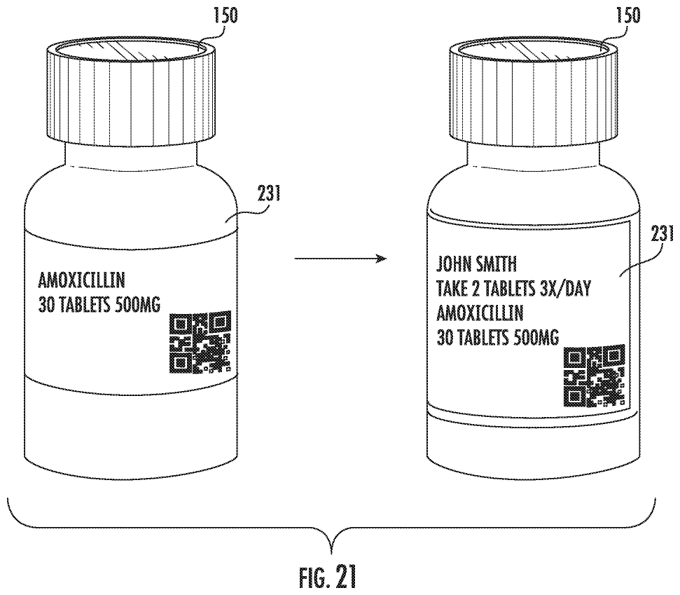

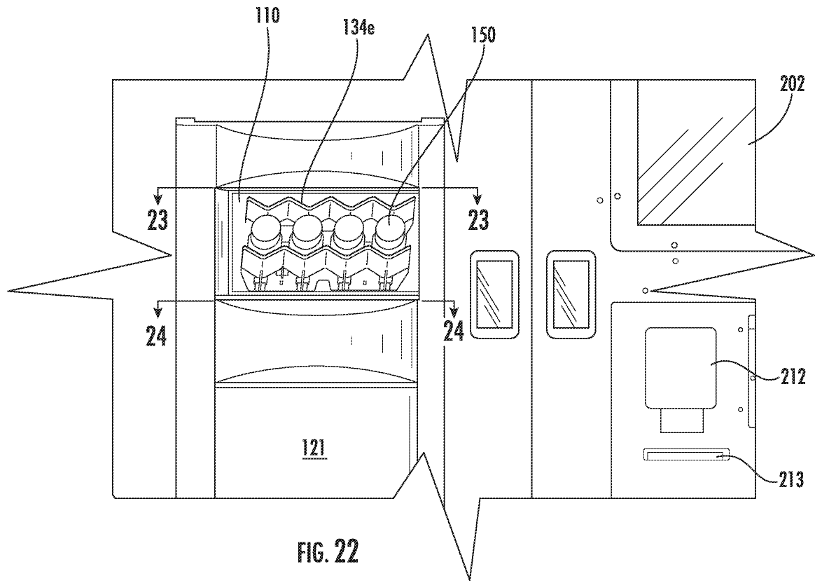

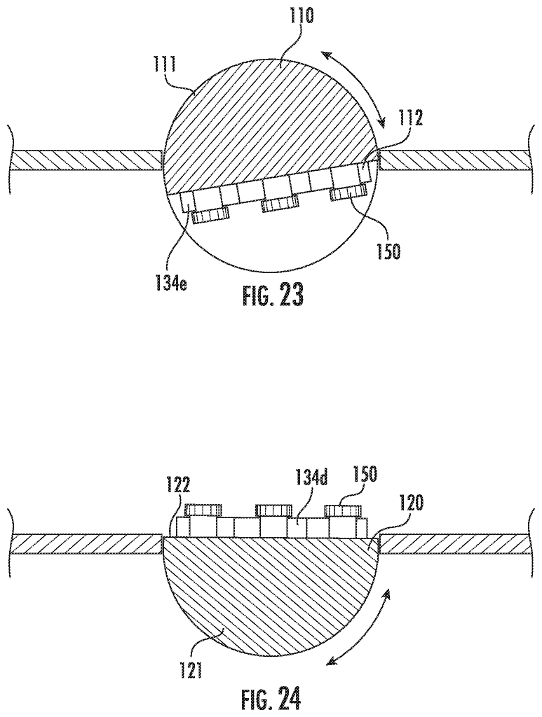

Label generating device (230) allows kiosk (50) to store containers of various medications in predetermined amounts and dosages, but upon dispensing, allows for the individualized labeling of the container specific to the user. For example, the label generating device (230) allows for a selected container to be dispensed for an authorized and verified user and have it labeled for the specific patient and the specific order. The information printed on the label may include, but is not limited to, the user/patient's name, the person prescribing the medication, type of medication, dosage information, instructions for taking the medication, hazards and warnings associated with such medication, as well as any transaction details. It is also within the scope of the invention to print details on the label as required by applicable regulations. This includes, for example, details that are required to be printed on the label as part of the transaction, as may be required in connection with the sale of cannabis. In sum, labeling generating device (230) may be used to print any desired information on the label and nothing herein is intended to limit the information which may be printed upon the label. As noted in FIG. 21, various bar codes or QR codes can be applied to bottle (150) or label (231) as needed.

In at least one embodiment, the container labeling station (136) includes at least one wheel operated by at least one motor. In these embodiments, a motive means, such as a robot arm (146), is intended to select, move and place a container (150) to be held in the designated container labeling station (136), by a shelf comprised of predesignated slots configured to hold the containers (150) or some other container holding means, including cubicles, compartments, bins or racks. A label is passed through one or more printers for providing the container and user specific information. The label includes an adhesive or other means for attaching the label to the designated container (150). The label is fed to the one or more wheels which rotate to affix the label to the one or more containers (150) of medication prior to dispensing. While one embodiment of the container labeling station (136) is provided, it is appreciated that many forms of automated container labeling exist in the art, and thus nothing herein is intended to limit the invention to any particular container labeling means.

### Pharmacy Vault

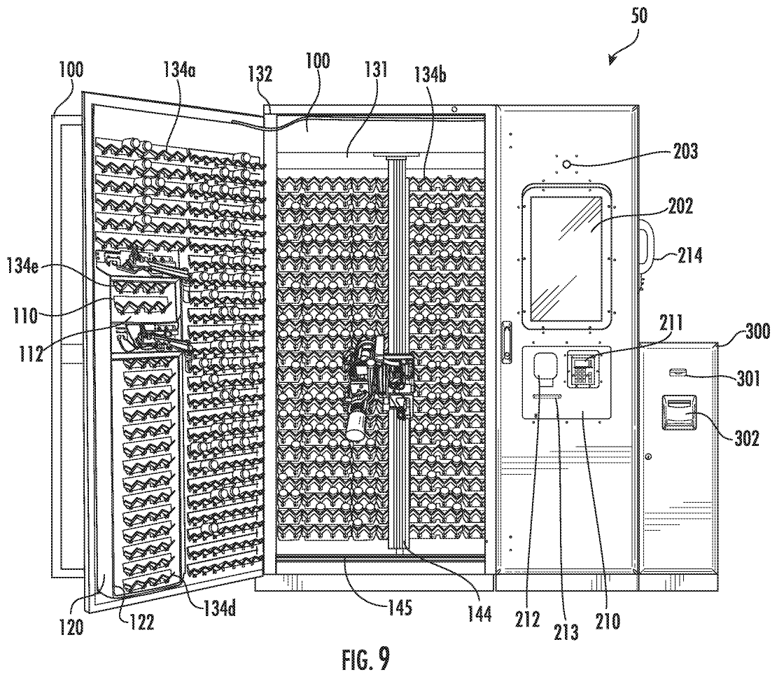

As illustrated in FIG. 9, opening the pharmacy vault door (130), reveals the interior of the pharmacy vault (100). This interior is defined by a pharmacy vault rear wall (131), a pharmacy vault external side wall (132), and an internal side wall (133) (common wall of the internal side wall (223) of the system vault (200)). A track system, including both vertical (144) and horizontal (145) tracks, is positioned within the interior of the pharmacy vault (100). These tracks support a robotic system as described in greater detail hereinafter.

The interior of pharmacy vault (100) houses a number of different shelves (134), each of which is configured to support and store an individual container (150). These containers (150) are stored at specified locations within vault (100) and can be accessed by a robotic system (140) that is movably positioned within vault (100). CPU (224) preferably includes an index of the specified locations to maintain an inventory of associated medicine containers (150). A first series of storage shelves (134a) are located on an interior surface of pharmacy vault door (130) and a second series of storage shelves (134b) are secured to rear wall (131) of the pharmacy vault (130). Additional storage shelves (134c) are mounted on the back wall of the system vault (200). Storage shelves (134a, 134b, and 134c) collectively make up a container holding portion (137). The pharmacy vault door (130) further comprises of a prescription loading portion (120) and a prescription dispensing portion (110), both of which are described below.

### Robotic System

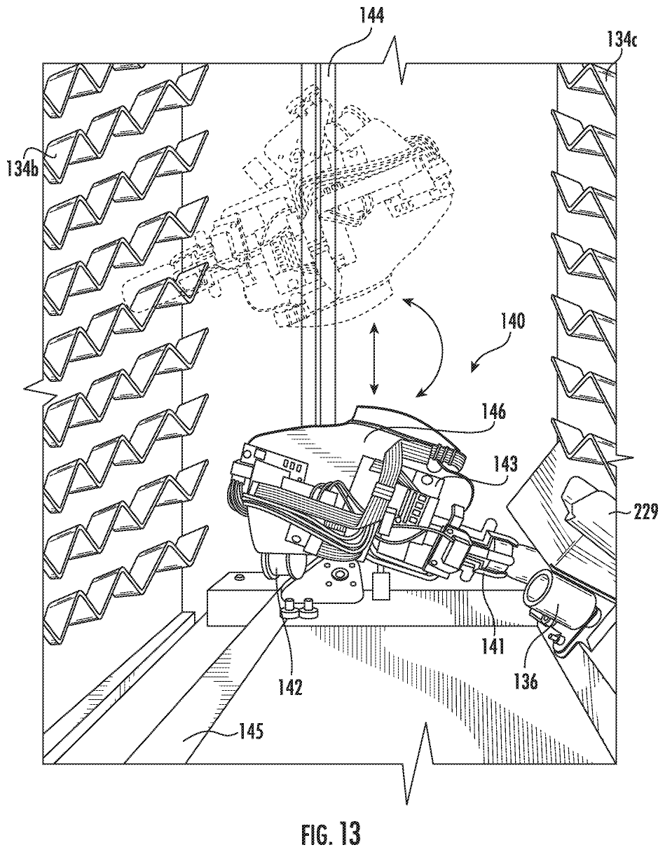

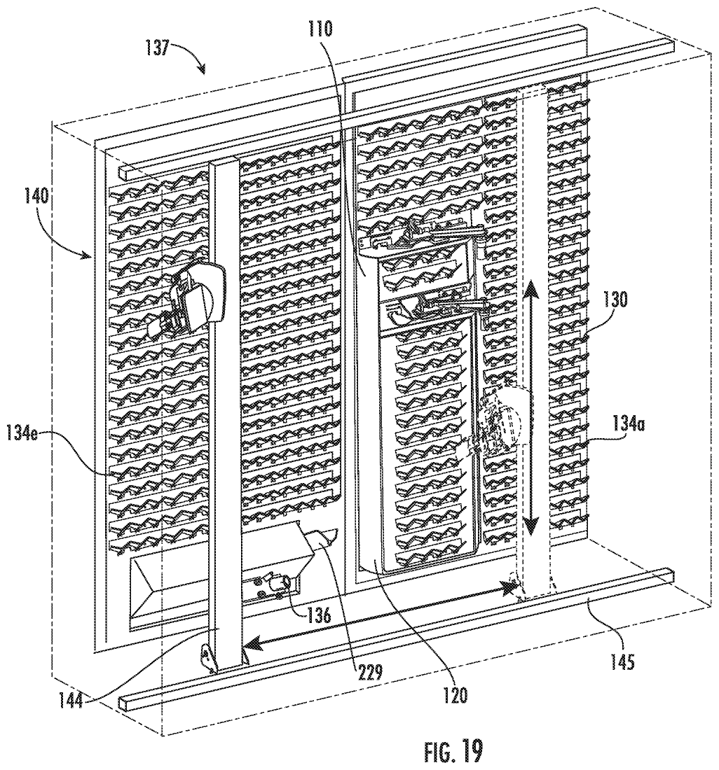

A robotic system (140) for accessing and handing the various containers (150) is located within the pharmacy vault (100). The robotic system (140) includes a bottle gripper (141), a motor (142), a scanner (143), and a robot arm (146) all of which travel along the vertical and horizontal tracks (144 and 145) within the pharmacy vault (100). Robotic system (140) can move up and down the vertical axis of vertical track (144) and along horizontal axis of the horizontal track (145). Additionally, and as shown by the arrows in FIG. 13, robotic system (140) can articulate in many different directions, including rotating on its base, to reach different sides and locations within pharmacy vault (100). Robotic system (140) can also be extended along and rotate about a central axis. This allows gripper (141), which is part of the robotic system (140), to reach any of the containers stored within holding portion (137) of the vault (100). Robotic gripper (141) may also hold a container (150) within labeling station (136) while a label is being applied. With reference to FIG. 19, in some embodiments, container labeling station (136) includes two smaller rollers and one larger roller for frictionally holding a container (150) in place while the bottle gripper (141) rotates the container (150) to affix a label to the container (150). The labels applied to container (150) are printed by label generating device (230) within the system vault (200) and are fed through a slot within the back wall of system vault (200).

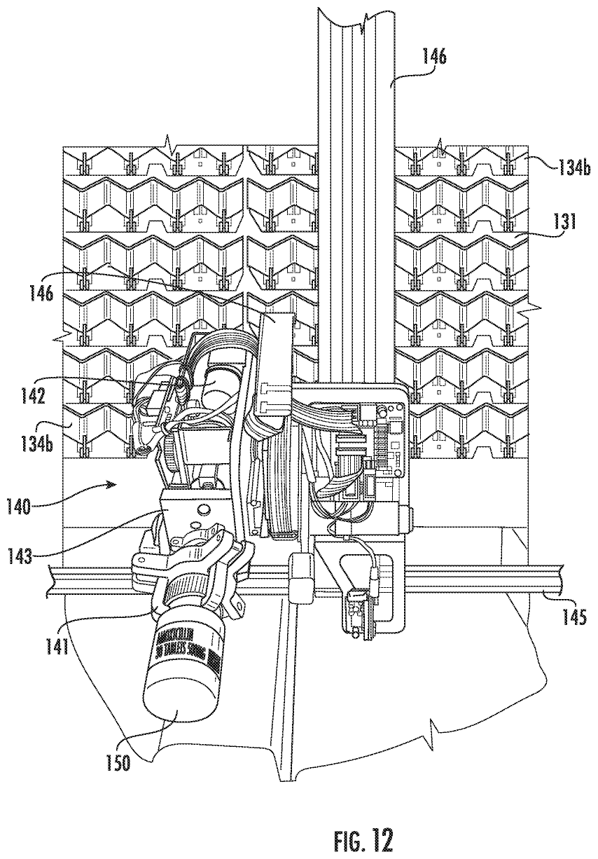

As illustrated in FIG. 12, a proximity sensor (147) is positioned adjacent to the robotic gripper (141). Proximity sensor (147) is employed in detecting whether a container (150) is present within a given location upon a V-shaped shelf (134). Sensor (147) can be employed in conducting an inventory of the pharmacy vault (100) or can be used to record the location of a specific container (150). Robotic system (140) also includes a camera (148) for taking photographs of containers (150) held by the bottle gripper (141). A photograph of an unlabeled container (150) can be taken immediately after the container (150) is loaded into the kiosk (50). This photograph may be used for purposes of inventory control. Thereafter, as a specific container (150) is being dispensed, camera (148) can be used to photograph the container (150) both before and after it has been labeled. These photographs, which can be viewed remotely by a pharmacist, can be used for verifying that the correct medication is being dispensed to the identified patient. Information about the medicine, in the form of a bar code, can be read by camera (148). As part of this verification, gripper (141) will retain contact with container (150) as both verification photographs are taken and gripper (141) will maintain such contact until the now verified container (150) is positioned within dispensing shelves (134e).

Robotic system (140) is also used in order to maximize volume utilization and inventory control, as well as, provide appropriate security and controlled access to the controlled substances. In at least one embodiment the robotic system (140) of kiosk (50) includes one or more robotic systems (140) for moving robotic arm (146) between predetermined locations and communicating mechanically with the container loading portion, container dispensing portion, and container holding portion based on commands from robot command software loaded within CPU (224). The robotic system (140), where used, allows for the movement of the containers between the various portions of the system that hold the controlled substance containers. In addition, use of robotic system (140) allows kiosk (50) to operate as a fully automated machine, in that, controlled substances may be stored in the container holding portion (137), and selected and dispensed for each user, without the need for human intervention or confirmation.

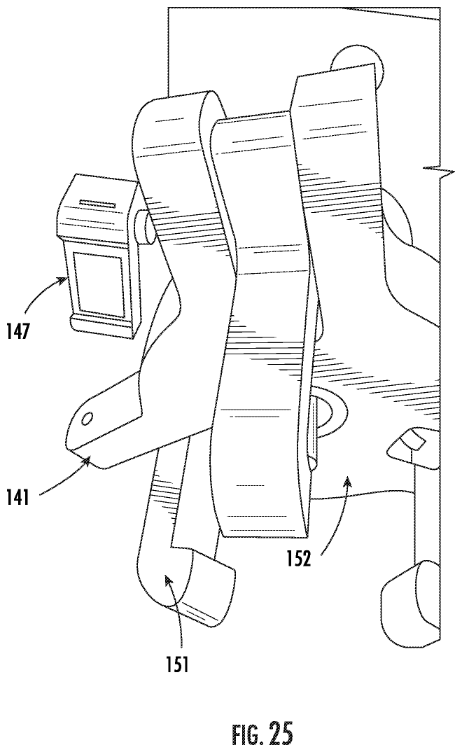

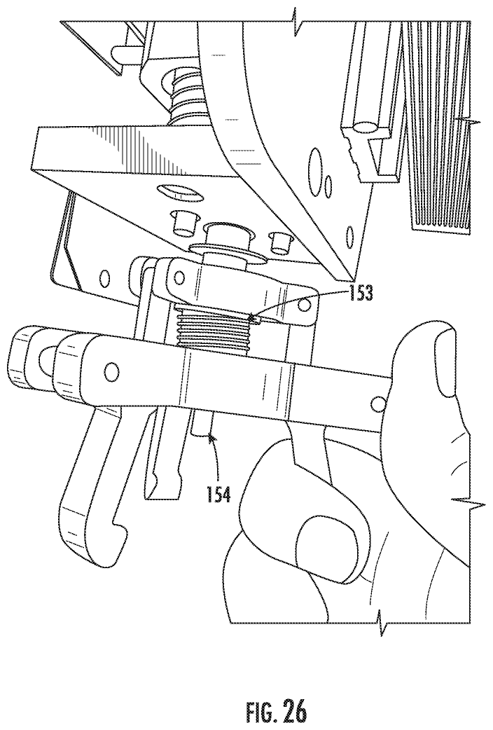

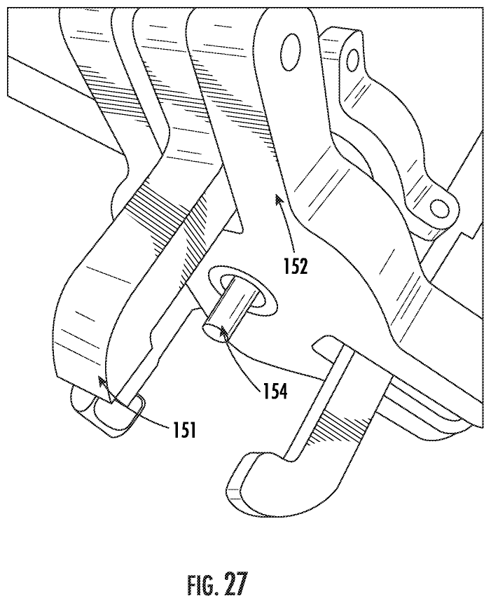

FIGS. 25-27 are more detailed views of gripper (141). As illustrated, gripper (141) comprises a housing (152) with a number of radial slots for supporting a plurality a fingers (151). In the depicted embodiment, three fingers (151) and three corresponding slots are provided. However, different numbers of fingers (151) can be employed. Fingers (151) move within slots between opened and closed positions. As illustrated in FIG. 26, a coil spring (153) is arranged between housing (152) and fingers (151) and functions in urging fingers (151) into a closed, gripping position. The bias of spring (153) can be overcome to thereafter open fingers (151). Solenoid actuators can be used in opening and closing gripper (141). FIG. 27 illustrates fingers (151) in the closed, gripping orientation and FIG. 26 illustrates fingers (151) in the opened, non-gripping orientation. In the preferred embodiment, a central support rod (154) extends through housing (152) and spring (153). Support rod (154) can be configured to detect the top of a container (150), thereby informing system (140) that container (150) is secured by fingers (151).

Once a container (150) is secured by gripper (141), camera (148) can be employed to take one or more pictures of the outside of container (150). For security reasons, a series of pictures are preferably taken of the container (150) during the loading and dispensing sequences described herein. Gripper (141) can also spin the container (150) to ensure that any barcodes are property read and to further ensure that any associated labels are photographed in their entirety. FIG. 25 also illustrates proximity sensor (147) located adjacent to gripper (141). In a preferred embodiment, sensor (147) is a laser based proximity sensor. It can be employed in recording the location of a container (150) within the pharmacy vault (100). It can likewise be used to detect the edge of a container (150) to thereby ensure proper alignment of gripper (141) over the top of the container (150). Radio Frequency Identification (“RFID”) can also be employed within vault (50) to keep a real-time inventory of containers (150). Namely, RFID tags can be applied to each individual container (150). This would allow an associated reader to record an individual container (150) entering or exiting the vault (50). Furthermore, ultra-high frequency tags can also be employed to allow the entire contents of the vault (50) to be recorded at any given time.

### Loading and Dispensing Portions

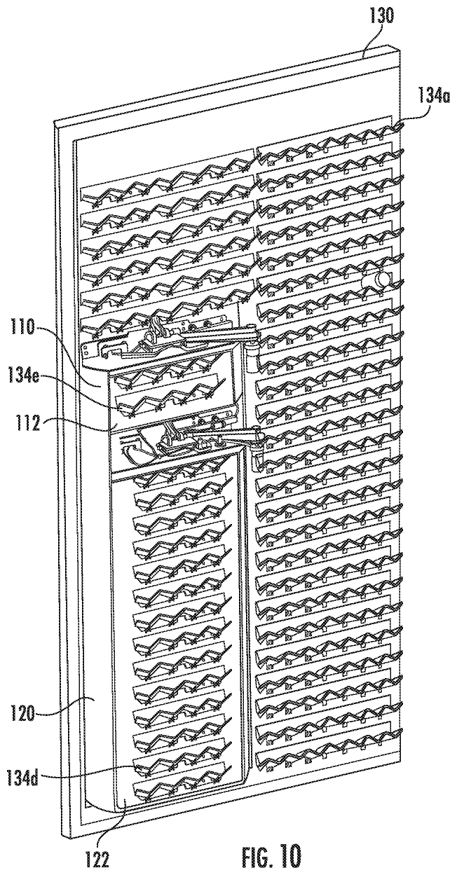

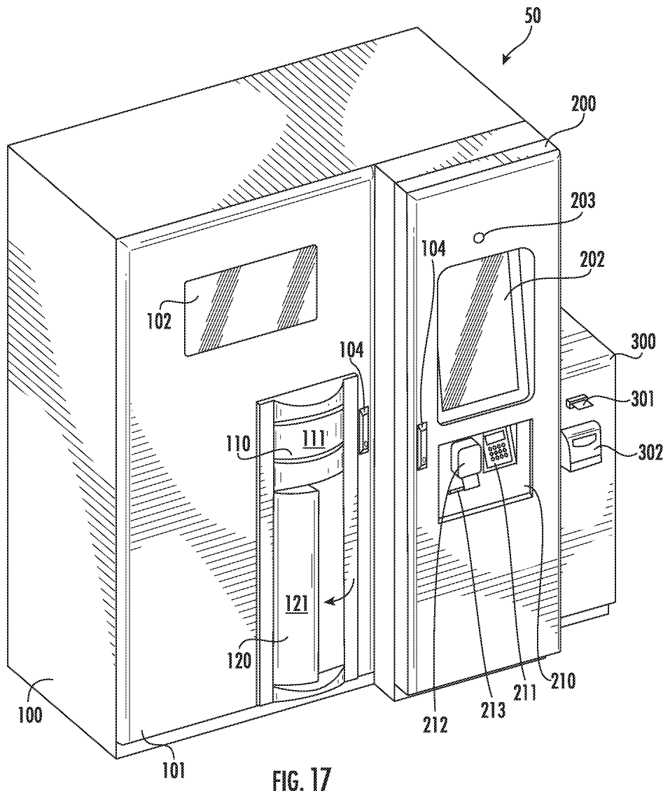

The container loading portion (120) is described in connection with FIGS. 10 and 17. In one embodiment, loading portion (120) is pivotally secured within the pharmacy vault door (130) and is accessible through an opening in access door (101). Loading portion (120) can be selectively rotated between secure and dispensing sides. V-shaped loading shelves (134d) are mounted to a flat interior side (122) of the loading portion (120). The, opposite, or exterior side (121), is preferably arcuate (or semi-circular) in shape and precludes access to the interior of pharmacy vault (100) when facing forward. It is also possible for exterior side to be generally flat with arcuate sides. With interior side (122) facing forward, loading shelves (134d) can be accessed from outside of kiosk (50) to thereby allow authorized personnel to stock the kiosk (50) with one or more medicine containers (150). When loading is complete, loading portion (120) is rotated so that exterior (or secure) side (121) is facing forward. In this orientation, shelves (134d) are open to the interior of the pharmacy vault (100) and accessible by robotic systems (140). Although loading portion (120) is preferably located within the pharmacy vault door (130), it is also within the scope of the present disclosure to position the loading portion (120) within the back of the kiosk (50). In such an embodiment, the exterior side (121) would be flat as opposed to arcuate.

Above container loading portion (120) is a container dispensing portion (110), which operates in a fashion similar to the loading portion (120). Specifically, dispensing portion (110) is pivotally secured within the pharmacy vault door (130) and is accessible through an opening in the pharmacy access door (101). In a preferred embodiment, both the loading portion (120) and dispensing portion (110) rotate about a central, vertical axis and have two functional sides. Both the loading portion (120) and the dispensing portion (110) can be selectively rotated between secure and dispensing orientations, with an arcuate exterior side corresponding to the secure orientation and a planar interior side corresponding to the dispensing orientation. Interior side (112) of dispensing portion (110) preferably includes a plurality of horizontally mounted v-shaped dispensing shelves (134e) that can be used to hold and dispense one or more containers (150) to a patient standing in front of the kiosk (50). Although FIG. 16 illustrates two rows of dispensing shelves (134e), any number rows can be employed. The exterior side (111) is preferably arcuate (or semi-circular) and secures the interior of pharmacy vault (100) when facing forward. This prevents the unauthorized access to the interior of pharmacy vault (100). When the dispensing portion (110) is in the secure (or shut) orientation, dispensing shelves (134e) are open to the interior of the pharmacy vault (100) and are accessible by robotic system (140).

The use of rotatable doors by the loading and dispensing portions (100 and 120) prevents the use of tools, hands or other means from accessing the interior of kiosk (50). It is believed that the use of the circular and rotatable door provides the required level of security as a controlled substance is being loaded or dispensed, in that the interior of the kiosk (50) is never exposed, even temporarily, to the exterior in a manner that would allow access by users or personnel not authorized to handle the controlled substances as it rotates, thus preventing access to the controlled substances by unauthorized users. Although the use of rotatable doors is described as preferred, it is within the scope of the present invention to employ doors that side between secure and dispensing/loading orientations. In this case, the dispensing portion (110) and loading portion (120) could each be slid into a secure orientation that precludes access to pharmacy vault (100). Thereafter, the dispensing or loading portion (110 and 120) could be slid downward to allow access to the dispensing or loading shelf.

Container storage shelves (134a, 134b, 134c), loading shelves (134d), dispensing shelves (134e), and container labeling station (136) are all in mechanical communication with each other by virtue of robotic system (140). Namely, robotic system (140) is adapted for gripping and moving containers within pharmacy vault (100). Robotic grippers (141) can access any of the containers (150) within pharmacy vault by traveling along vertical and horizontal tracks (144 and 145) and by pivoting to either side. This allows robotic system (140) via grippers (141) to transfer containers (150) from loading shelves (134d) to the first and second storage shelves (134a and 134b) within pharmacy vault (100). In some embodiments, containers (150) are also be stored upon shelves (134c) positioned on the back side of system vault (200), with shelves (134c) likewise being accessible by robotic system (140). Once an order is confirmed, robot (140) via grippers (141) may move a particular container (150) to labeling station (136) where an appropriate label is applied. Thereafter, the labeled container (150) can be positioned within upon one of the dispensing shelves (134e). Once the identity of the patient is verified, the dispensing portion (110) can thereafter be rotated such that the labeled container (150) is presented to the patient.

### Kiosk Operation

To load pharmacy vault (100), a number of prepackaged containers (150) are brought to kiosk (50) by an appropriate representative, such representative being a pharmacist or other authorized handler of prescription drugs. A pharmacist or other authorized handler approaches the kiosk (50), and selects on the user interface (202), whereby the identity of the authorized representative is confirmed. The representative may enter a code into interface (202) or their identity can be confirmed via video camera (203). The appropriate level access to kiosk (50) is also determined at this point. Other verification methods that may be standard within the medical industry may also be employed. This includes scanning a credential (such as a driver's license or ID card) or via any of a variety of biometric identification means. The door to the container loading portion (120) is unlocked and rotated, exposing the shelves (134d) of the container loading portion (120) to the exterior of the device (50). The pharmacist or other prescription drug professional loads the containers (150) in the container loading portion (120), and indicates at the user interface (202) when completed. The inventory software of CPU (224) may be updated to reflect the quantity, type, and location of the medicines being supplied. The door to the container loading portion (120) rotates closed and locks, such that shelves (134d) and containers (150) are now facing the inside of the pharmacy vault (100).

Thereafter, robotic system (140), based on commands from CPU (224), moves the containers (150) from shelves (134d) of container loading portion (120) to storage shelves (134a) and (134b). In some embodiments, containers (150) are also loaded onto storage shelves (134d), as well as other locations within holding portion (137). These indexed locations of the medication within the holding portion (137) are recorded by the inventory software of CPU (224).

When a user/patient arrives at kiosk (50) to retrieve their prescription, the patient interacts with the user interface (202), and one of the keypad (211), credit card reader (212), or cash payment systems (302) to provide any necessary payment required for retrieving their prescription. CPU (224) with its associated software communicates with a central processing system (CPS) (422) (described below) to verify the patient and that the patient is authorized to receive the identified medication. This may include determining that the patient is the appropriate age for the medication and that the identified medication can be lawfully dispensed in the relevant geographic area. CPS (422) can also be employed in verifying the patient's insurance coverage and computing any payment that may be necessary. Video camera (203) can thereafter be used to verify the identity of the patient. Alternatively, the identity of the patient can be confirmed by having the patient enter an appropriate code into interface (202), which such code being assigned to the patient by the prescribing entity. Once the user/patient has been authenticated, the insurance information confirmed, and the prescription authorized, CPS (422) issues a command to CPU (224) to dispense the appropriate containers (150).

Robotic system (140) thereafter retrieves the appropriate containers (150) from the container holding portion (137) for that patient's prescription. Robot system (140) selects and moves the container (150) to the container labeling station (136) where one or more containers (150) are labeled for the specific order. The particular container (150) is then moved to a shelf (134e) of container dispensing portion (110). Once all the containers (150) for a particular prescription have been retrieved, labeled and delivered to the container dispensing portion (110), container dispensing portion (110) is unlocked and rotated so that interior side (112) with shelves (134e) are presented to the user and they may retrieve their prescription(s).

Robotic system (140) has a scanner (143) that allows it to determine whether a certain container (150) is present anywhere within the pharmacy vault (100). Robotic system (140) can also move containers (150) to predetermined locations as referenced in the inventory database for kiosk (50). This function allows for determining if someone has received their prescriptions after the system has timed out and the dispensing portion (110) has been closed or the customer canceled an order. Furthermore, this function can eliminate the attempt of the robotic system (140) from grabbing improperly inserted items and blockage items. One the kiosk system dispenses the appropriate containers (150), inventory software of CPU (224) is updated. CPU (224) also communicates with CPS (422) to update the centralized inventory databases. CPS (422), as needed, can initiate refill requests when the inventory levels reach a certain predetermined level.

Kiosk (50) can also be employed in discarding unwanted containers (150). These may be containers (150) that are not picked up by patients, are recalled, or otherwise expire. In any case, CPU (224) initiates a command to move robotic system (140) from one location to one or more predetermined locations where the unwanted containers (150) are located. Thereafter, the picking arm (146) and bottle gripper (141) of robotic system (140) can be used to put the unwanted container (150) into a discard slot that extends through the interior wall (133) of the pharmacy vault (100), through the interior wall (223) of systems vault (200), and into a secure discard container (227). This allows unwanted containers (150) to pass from the pharmacy vault (100) to the secure container (227) within system vault (200). In another embodiment, discard container (227) is accessed from a discard slot that is formed within a rear wall (221) of system vault (200). This discard slot (229) is likewise accessible from the interior of the pharmacy vault (100). In either event, a technician can access, via the system vault (200), the locked container discard station (227) without accessing the pharmacy vault (100). This separating of the pharmacy vault (100) from the system vault (200) provides additional security and maintains proper regulatory safeguards. The kiosk system software of CPU (224) also communicates with CPS (422) to update kiosk inventory levels when robotic system (140) moves an expired or discarded controlled substance to the container discard station (227).

### Remote Prescription System

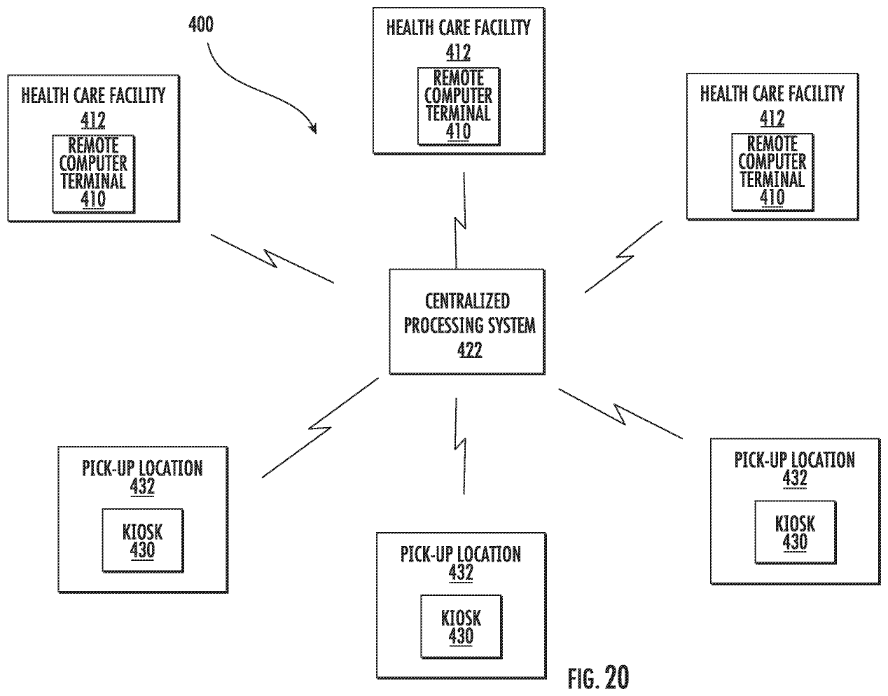

System (400) of the present disclosure is described next in connection with FIG. 20. FIG. 20 illustrates how a kiosk, such as master kiosk vault (50), can be employed as part of a remote prescription system (400) that allows health care professionals to remotely prescribe medications, such as controlled substances, to a patient. These patients may then retrieve the prescription at one or more pick-up facilities. System (400) also allows a particular medicine container to be automatically labeled and dispensed at the pick-up facility to the specified patient. The various components of system (400) are described below.

System (400) employs one or more remote computing stations, or remote computer terminals (410) (“RCT”). An RCT (410) may be located, for example, in a doctor's office, hospital, clinic, or anywhere else a medicine or substance may be prescribed for a patient. RCT (410) can be used to view, access, and generate electronic health records (EHRs) for a particular patient. FIG. 20 illustrates the RCT (410) located within a health care facility (412). RCT (410) can be used by a pharmacist, doctor, nurse, nurse practitioner, physician's assistant, or any other individual who may be prescribing a medication to a patient. It may likewise be used by assistants or technicians who may be assisting the individual prescribing the medication. In any event, the health care provider uses the RCT (410) to enter prescription information for a specified patient into system (400). Multiple RCTs (410) at multiple facilities (412) can be employed by the system (400). Similarly, multiple kiosks (430) at multiple pick-up locations (432) can also be used by system (400).

The prescription information may include, for example, the type of medicine being prescribed, the amount of medicine within the container, the dosage for the patient, the expiration date, as well as any instructions. By way of the RCT (410), the health care provider may also input various details regarding the individual patient. These details may include, but are not limited to, the patient's name, age, address, preferred pick-up location, and health insurance information. Other information for identifying the patient can also be supplied, such as a driver's license number, social security number, or birthdate.

RCT (410) is in remote communication with a centralized processing system (422) (“CPS”). CPS (422) may be a computer server, a computer cluster, a server pool, a general-purpose personal computer, a workstation, a laptop, a series of networked hard drives, or any other type of computing device that utilizes a storage medium for securely storing digital information. As used herein, “processing system” is intended to compass all of these various computing devices. Any information entered into RCT (410) can subsequently be uploaded and stored in the CPS (422). For example, the health care worker can upload to CPS (422) the prescription information and the patient details. All such information is securely housed within CPS (422) for subsequent retrieval.

CPS (422) ideally runs any of a variety of prescription management and inventory software. This allows CPS (422) to verify details regarding a particular prescription or a specified patient. Namely, CPS (422) ideally has access to one or more databases, which may be resident within the CPS (422) or remotely accessed. These databases allow CPS (422) to verify: the identity of the specified patient; that the patient is authorized to receive the prescribed medicine; and as well as the patient's health insurance information and benefits. CPS (422) can also be employed to in verifying that the medicine type is available at the identified pick-up location. One or more third parties may be involved in these verification steps. The verification steps may be carried out prior to the patient arriving at a kiosk to pick up a prescription, thereby allowing the patient or health care provider to be alerted of any problems in fulfilling the order. Alternatively, these verification steps may be carried out only after the patient arrives at the kiosk.

With continuing reference to FIG. 20, the kiosks (430) that form part of system (400) are described. As illustrated, one or more kiosks (430) can be located at an identified pick up location (432). Additionally, multiple pick-up locations (432) can be part of system (400). As used herein, a pick-up location may be, for example, a super market, drug store, pharmacy, or anywhere else a patient may go to pick up medication or a prescription. It is also within the scope of the present invention to position a kiosk (430) at a location that is not traditionally associated with picking up prescription medication. For example, kiosk (430) can be located at a convenience store, gas station, or movie theater. The present invention is not intended to be limited to any particular location for kiosk (430).

Kiosk (430) may include any of the features described above in connection with kiosk (50). For example, kiosk (430) may include a pharmacy vault (100) for storing a plurality of medicine containers and a robotic system (140) for handling the plurality of medicine containers (150). Kiosk (430) may also include a system vault (200) with a central processing unit (224) (CPU) that is in remote communication with the CPS (422). This allows CPU (224) to download the prescription information and patient details for the specified patient to a kiosk (430). System vault (200) also includes a user interface (202) and video camera (203) for allowing the identity of the specified patient to be verified.

The use of user interface (202) affords the opportunity for a particular user/patient to interact with kiosk (50) to, among other actions: have their identity authorized and authenticated; to receive prescriptions called in to ensure that the controlled substances are being dispensed to the correct person; select the controlled substances to be dispensed; provide order information for a pre-ordered controlled substance; provide payment information; or any other interactions for the automated dispensing of controlled substances. User interface (202) may also allow for the interaction of authorized substance handler personnel to access the container loading portion for the restocking of controlled substances to kiosk (50). User interface (202) may also allow for key-less, electronic access to the interior of the kiosk (50) by appropriate individuals by utilizing digital access codes to unlock the electronics vault or controlled substance vault, as appropriate.

In use, label generator (230) (which is positioned within system vault (200)) creates a label containing the prescription information for the particular medicine container (150). Labeling station (136) thereafter functions to apply the label to the particular medicine container (150) and, thereby, create a labeled container that is ready to be dispensed to the patient. Labeling station (136) is accessible by robotic system (140). The labeled container (150) can be stored within the pharmacy vault (100) until it is ready to be dispensed. A dispenser (110) is secured within the pharmacy vault (100) and is utilized in dispensing a labeled container (150) to the patient. The dispenser (110) includes a secure side (111) and a delivery side (112) with a shelf (134e). Dispenser (110) is selectively rotatable between the secure (111) and delivery sides (112). Robotic system (140) is adapted to transfer the labeled container (150) to the shelf (134e) upon the identity of the specified patient being verified by CPS (422). Dispenser (110) is thereafter rotated to the delivery side (112) to allow the specified patient to retrieve the labeled container (150).

System (400) and kiosk (50 or 430) can be configured with multiple means for user interaction, including through manual interaction (such as a keyboard or touch pad), mobile app, electronic or digital communication, an authentication card, biometric identification, using a smart phone or other handheld device and other means for communicating. In some embodiments a phone (214) may be attached to the exterior of the master kiosk vault (50), to provide privacy when communication or video chatting with the pharmacist, alternatively speakers may be inserted into the headphone jack, or a user/patient could connect to the kiosk (50) via Bluetooth or through other wireless speaker devices.

### Dispensing Method

The present disclosure further provides for an automated method for the dispensing of controlled substances at remote locations. In one embodiment of the invention, the method includes one or more remote computing locations for initiating, ordering and/or uploading information to allow for the dispersal of one or more controlled substances to a user/patient upon the user/patient interacting with the user interface. Upon the authorization and authentication of a user/patient by the third party authorization and authentication provider, the kiosk provides a method of supplying a user/patient with a controlled substance through completely automated, robotic means, without the need for human interaction or supervision.

In at least one embodiment of the invention, upon authentication and authorization of a user/patient, a method for dispensing the controlled substance to the user/patient is initiated. A robotic system housed inside of the pharmacy vault where the inventory of controlled substances is stored, in a fully automatic process, selects the controlled substances that match the prescription ordered by the patient/user from the container holding portion of the vault, and then labels the one or more containers with the patient/users specific identification information, instructions for use, dosage, and other information appropriate and commonly included on prescription medication bottles. Following the labeling of the one or more containers, the robotic system moves the prescription container into the shelving slots of the revolving container dispensing portion. The revolving container dispensing portion, rotates from the interior of the pharmacy vault of the device to the exterior of the device, such that the user/patient can then access and pick-up their one or more prescriptions.

### Additional Embodiments

In at least one embodiment, one or more specific containers of a controlled substance may be pre-labeled in anticipation of an order fulfillment by a particular authorized and authenticated user. In embodiments where a kiosk is associated with a system according to the invention, external processes may direct one or more kiosks to prepare a controlled substance for a particular user, whereby, a controlled substance is labeled by the container labeling station prior to a user/patient requesting the controlled substance. In such embodiment, the pre-labeled medicament may be stored in the container labeling station, the container holding portion, or any other place within the kiosk that may have a shelf or other means for holding a container prior to dispensing.

In alternative embodiments, a user/patient may be able to seek pre-authorization and authentication, before visiting the kiosk, such that if they visit the kiosk after-hours, to pick up a prescription, they will be able to pick-up their prescription without having to directly communicate with the pharmacist through the user-interface at that time.

It is appreciated that some instances one or more containers of a controlled substance may need to be disposed of, or taken out of circulation, prior to the time authorized loading personnel for the kiosk are available. In at least one embodiment, the kiosk includes a means for discarding certain designated containers. For example, in some embodiments, where a means for discarding specific containers is employed, the mechanical moving means, such as a robot, will select one or more designated containers that need to be discarded and deposit such containers into a discarded container station. A discarded container station may include a storage station, such as a separate series of shelves or other designated container holding spaces, located within the kiosk that are later unloaded when authorized handling personnel approach the kiosk to load or maintain the kiosk. In some embodiments, a discarded container station may be a locked container with a slot positioned and sized for receiving discarded containers being discarded from the system. It is appreciated that reasons for discarding containers may vary, and nothing herein is intended to limit reasons why a container is transferred to the discarded container station.

Those skilled in the art should readily recognize that because of the use of automated means and communications systems that the inventive kiosk will require one or more means for supplying power to the various sections or aspects of the kiosk, including, without limit, the motive means, communication system, container labeling portion, and user interface. All types of power can be used including direct power, solar and wind power, batteries, generators and energy storage systems. This would allow for the devices to be placed in a variety of remote locations including remote campuses and positions such as mobile hospitals in military applications. Nothing herein is intended to limit the mode or method for supplying power to one or any of the components of the kiosk.

Those skilled in the art should also readily recognize that the storage of controlled substances, and the use of various electrical components, may require one or more means for cooling or warming the interior of the kiosk, in order to maintain the appropriate temperature and humidity controlled environment, necessary to preserve the integrity of the controlled substances. All types of systems can be used including thermoelectric cooling systems, with adjustable thermostats depending on the environment in which the kiosk has been placed. Nothing herein is intended to limit the mode or method for cooling, heating or maintaining the temperature and environment within the kiosk.

In some embodiments, the pharmacy vault door and the system vault door are additionally secured by a common vault door that secures over each. Where used, it is appreciated that a common vault door may have one or more apertures for components of the pharmacy vault door or system vault door, including, without limit, to allow for the container dispensing portion or container loading portion of the first pharmacy door, or a user interface if attached to the exterior of the system vault door.

Because inventive embodiments of the kiosk described herein may be used in association with one or more systems for the automated ordering and dispensing of controlled substances, some embodiments of the kiosk have been incorporated in to one or more systems.

The one or more remote computing locations for initiating, ordering and/or uploading information provide for the functionality of remote initiating, ordering and/or uploading information. Nothing herein is intended to limit the remote initiation, ordering and information uploading methods to anything less than is what known in the art. In addition to the known methods, in some embodiments of the inventive system, the remote ordering is specific to a controlled substance. For example, where used for the administration of prescription medicaments, the remote ordering may be through prescription ordering software accessed by a doctor or other medical personnel to initiate, order and/or authorize the dispensation of one or more prescription drugs for a patient to treat one or more particular conditions. Information that may be uploaded is patient information, insurance information, or a person's address to help determine the most convenient location for the dispensation of the controlled substance. It is appreciated that such software, when used in the dispensation or administration of prescription drugs, may include communications with one or more electronic medical records, as well as communications with insurance software to determine reimbursement and payment information for a particular patient, where such information may be distributed to the kiosk for a patient to remit payment at the time of dispensation of the controlled substance.

The central processing system (“CPS”) communicates with the remote computing locations and the kiosks to link the ordering process with the dispensing process, thus allowing for the automated and remote dispensation of controlled substances. Generally, one aspect of the invention includes the ability for a user to conveniently access their orders, through a user friendly interface. Thus, in at least one embodiment, the inventive central processing system includes inventory management and location information such that the system can instruct a user as to the availability and locations of the controlled substance so that the controlled substance may be retrieved at their convenience. In such embodiments, the central processing system updates and maintains a database of available controlled substances for each kiosk connected to the system and communicates with such kiosks to maintain updates to the database in real-time. By providing this, a person who is ordering or uploading an order to the system may be able to select the kiosk location at which the controlled substance is to be received, provided that the ordered controlled substance is available at that location in the ordered quantity. It is further appreciated that in some embodiments, where a particular controlled substance is available in an increment more or less than what was ordered, that a user may be prompted to change or split an order so that the order may become available, at a more convenient kiosk.

The inventive central processing system includes inventory management software that allows the robotic system within the kiosk to monitor the inventory within a kiosk and provide updates to the central processing system in real-time, to compare and control inventory levels. The automatic inventory update is sent in real-time, from the kiosk to the central processing system, in order to avoid a user having to travel to a specific kiosk, and then find out that a specific controlled substance is unavailable. When inventory volume reaches certain preset levels, the central processing system will send a command to the authorized company to refill a specific controlled substance, in order to decrease or prevent the unavailability of controlled substances.

The robotic system is further preprogrammed to scan controlled substance containers on a routine basis, to check for out of date prescriptions that have been stored for too long or have expired and remove them from the container storage portion and deposit them into the discarded container station that receives the unused or expired prescriptions.

No matter the reason for the removal of a controlled substance from the kiosk, the robotic system will provide an update to the central processing system of the new inventory levels. Regardless of whether the robotic system dispenses controlled substances to authenticated and authorized users/patients, or removes from the kiosk, expired or other discarded containers, the robotic system provides updates to the central processing system to track inventory levels. The central processing system sends alerts to the warehouse when inventory reaches certain preset levels, such that authorized handlers may come and refill the kiosk.

### Apparatus and Method for Automatically Dispensing Cannabis

The apparatus and method described above can also be employed in dispensing recreational or medically prescribed cannabis. More specifically, the master kiosk vault (50) will include a vault (100) with a series of v-shaped shelves. These shelves would include shelves on the vault door (134a), shelves on the vault wall (134b), and shelves on the back of the system vault (134c). As described above, each of these shelves could house an individual container (150). However, unlike the primary embodiments described above, container (150) would contain a volume of cannabis. This cannabis could be intended for either recreational or medical use. The remainder of the system would operate as previously described. Namely, the loading portion (120) with its v-shaped shelves (134d) would be employed in populating the vault (50) with filled containers. Likewise dispensing portion (110) with dispending shelves (134e) would be employed in dispensing containers (150) with cannabis to customers or patients. Additionally, the robotic system (140), with a bottle gripper (141), could access individual containers (150) upon shelves (134) as described. Thus, all the components of the system described above can readily be employed in dispensing medical or recreational cannabis.

Nonetheless, some additional security features, described below, could optionally be incorporated into the system in view of the nature of the product being dispensed. Namely, the user could be prompted to insert a government issued identification via card slot (301) in side car vault (300). A scan of the card would then be provided to a remotely located third party for the purpose of indicating whether the proffered identification was fake or genuine.

If the identification is valid, camera (203) would then be used to confirm that a live user is present at the kiosk (50). This can be accomplished by prompting the user to blink or move their head. Provided a live user is present, system takes a picture of the user via camera (203) to confirm that the user matches the photograph in the presented identification. In this regard, a light ring may be provided about camera (203) to provide a clear and properly illuminated picture. The third party can, thereafter, provide a confidence rating, indicating the likelihood that the picture matches the live user present at the kiosk (50).

Still yet other security protocols can be provided. Camera (203) can be configured to detect whether the user is alone or with others. The system can thus be programmed to reject a sale if the user is not alone, thereby reducing the likelihood that an otherwise authorized user will make the purchase for an unauthorized user or minor. Further programming can be included to detect whether the user is under the influence. This can be accomplished, for example, by detecting whether the user is swaying, unsteady, or has blood shot eyes. The system, via interface (202), can also prompt the user to conduct drills to allow the system to assess the user's hand/eye coordination. In the event an impairment is detected, the transaction can be declined. One or more of the various security protocols described herein may be desirable when dispensing either recreational or medical cannabis. They could also be employed if the system is used to dispense dangerous medications or certain scheduled narcotics.

Provided the user passes the various security protocols, they will be permitted to proceed with the cannabis purchase. Namely, upon verification and authentication, user interface (202) will display a variety of different types of cannabis to the user. The variety of types of cannabis displayed may be based upon the user's geographic location or details gleaned from their presented identification. The system may also employ the user's identification, in conjunction with relevant government maintained databases, to confirm that the user has a prescription for medical marijuana. The database can also be consulted to verify the user's allotted daily or weekly limit. In this regard, a single kiosk (50) can be used to dispense both recreational and medical cannabis. The option to shop for either medical or recreational cannabis can be presented to the user at the outset of the transaction, or the decision can be automatically made by the system on the basis of information gleaned from the user's identification.

For either recreational or medical cannabis, the user is permitted to review the various cannabis types available for purchase. The user will also be able to follow on-screen prompts to obtain additional information about the various types of cannabis. This additional information may include the marijuana strain, the hybrid, the origin and geographic location of the plants, and THC levels. Information regarding the benefits of particular strains can also be provided, such as strains that promote relaxation, energy, or focus. User interface (202) can also allow user to interact with a pharmacist as may be required in certain jurisdictions.

A point of sale (“POS”) system can then be employed in completing the sale. Namely, once the user choses the type and amount of cannabis to purchase, the items are added to a virtual shopping cart that is visible at user interface (202). The user will then be prompted for payment, with payment being made via the credit card reader (301) or cash payment processing device (302) within side car (300). Some jurisdictions may require certain transaction details are printed on a label. In such instances, label generating device (230) will generate the necessary label (231) to be applied to container (150) prior to it being dispensed. Transaction details can optionally be emailed to the user or sent via text message.

Secondary facial recognition may also be conducted by camera (203) as a final verification of the user prior to container (150) being dispensed. The POS system may also communicate relevant transaction details to appropriate government databases to comply with necessary regulations, such as seed-to-sale tracking. As is known in the art, the POS system will likewise communicate transaction details to an inventory management software.

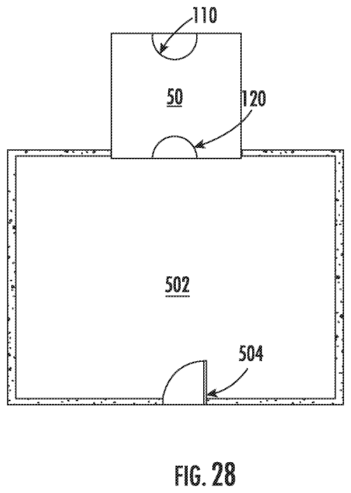

FIG. 28 illustrates an alternative layout for the kiosk (50). In this embodiment, kiosk (50) is connected to a facility (502) that may store a large volume of cannabis to be dispensed by system. Access to the storage facility (502) would be controlled by a secure access door (504). Door (504) can be provided with a secure coded lock or similar security features. This would allow personnel to access facility (502) for restocking of supplies. Personnel within facility (502) could then use a rotatable container loading portion (120) to load containers (150) into the vault. A user would then access one or more purchased containers (150) via rotatable dispensing portion (110). Dispensing portion (110) and loading portion (120) can be as described above in connection with the primary embodiment of the kiosk.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the described embodiments in any way. Rather, the foregoing detailed description will provide those skilled in the art with a convenient road map for implementing the exemplary embodiment or exemplary embodiments. It should be understood that various changes can be made in the function and arrangement of elements without departing from the scope as set forth in the appended claims and the legal equivalents thereof.

What is claimed is:

1. A device (20) for storing, labeling, and dispensing containers (150), at least one container (150) storing a controlled substance prescribed for a patient, access to the device (20) being limited to authorized individuals, the device (20) comprising:
a pharmacy vault (100) with an interior area, a pharmacy access door (101) and a vault door (130) each hingedly secured to the pharmacy vault (100) and each having opened and closed positions, the pharmacy vault (100) housing a vertical and a horizontal track (144, 145);
a system vault (200) with an interior area and a rear wall (221), a system access door (201) hingedly connected to the system vault (200) and having an opened and closed positions, the system vault (200) housing a central processing unit (224);
a first series of storage shelves (134a) secured to an interior surface of the pharmacy vault door (130), a second series of storage shelves (134b) secured within the interior area of the pharmacy vault (100), a plurality of containers (150) positioned upon the first and second series of storage shelves (134a and 134b);
a rotatable loading portion (120) pivotally secured within the pharmacy vault door (130) and accessible through the pharmacy access door (101), the loading portion (120) including an arcuate exterior side (121) and a planar interior side (122), the interior side (122) supporting a plurality of loading shelves (134d), the loading portion (120) being selectively rotated to permit access to the loading shelves (134d) by the authorized individual from outside the device (20);
a rotatable dispensing portion (110) pivotally secured within the pharmacy vault door (130) and accessible through the pharmacy access door (101), the dispending portion (110) including an arcuate exterior side (111) and a planar interior side (112), the interior side (112) supporting a plurality of dispensing shelves (134e), the dispending portion (110) being selectively rotated to permit access to the dispensing shelves (134e) by the patient from outside of the device (20);
a robot (140) secured within the interior area of the pharmacy vault (100) and adapted for movement along the vertical track (144) and the horizontal track (145), the robot (140) including a motor (142), a scanner (143), and a container griper (141) for gripping a container (150), the robot (140) adapted to transfer containers (150) from the loading shelves (134d) to the first and second storage shelves (134a and 134b) and thereafter from the first and second storage shelves (134a and 134b) to the dispensing shelves (134e);
a label generating device (230) positioned within the interior area of the system vault (200), the label generating device (230) including a supply of labels (231), a printer (235), and a take up wheel (232), the central processing unit (224) specifying the content of the labels, a labeling station (136) accessible through the rear wall (221) of the system vault (200), the robot (140) adapted to position containers (150) within the labeling station (136) whereby labels from the label generating device (230) can be affixed;
a discard station (227) secured within the interior area of the system vault (200), the discard station (227) being accessible through the rear wall (221) of the system vault (200), the robot (140) adapted to transfer unwanted containers (150) to the discard station (227).

2. A device for storing and dispensing medicine containers comprising:
a pharmacy vault with an interior area and a vault door;
a system vault with an interior area and a system access door, the system vault housing a central processing unit;
a series of storage shelves secured within the interior area of the pharmacy vault, a plurality of medicine containers positioned upon the storage shelves;
a loading portion positioned within the pharmacy vault door, the loading portion being selectively positionable between secure and accessible orientations, with the accessible orientation allowing containers to be loaded within the pharmacy vault and the secure orientation precluding access to the pharmacy vault.

3. The device as described in claim 2 further comprising a dispensing portion secured within the pharmacy vault door, the dispensing portion being selectively positionable between secure and accessible orientations, with the accessible orientation allowing containers to be dispensed from the pharmacy vault and the secure orientation precluding access to the pharmacy vault.

4. The device as described in claim 2 wherein the loading portion rotates between the secure and accessible orientations.

5. The device as described in claim 3 wherein the dispensing portion rotates between secure and accessible orientations.

6. The device as described in claim 4 wherein the loading portion includes a shelf for supporting a medicine container with the shelf being accessible from outside of the device when the loading portion is in the accessible orientation.

8. The device as described in claim 5 wherein the dispensing portion includes a shelf for supporting a medicine container with the shelf being accessible from outside of the device when the dispensing portion is in the accessible orientation.

9. The device as described in claim 2 further comprising a labeling station that is accessible through a wall of the system vault, and a robot adapted to position containers within the labeling station whereby labels from the label generating device are applied.

10. A system for remotely prescribing and automatically dispensing a particular medicine container to a specified patient, the system comprising:
a remote computing terminal (RCT) for use by a health care provider, the health care provider inputting into the RCT prescription information for the specified patient as well as patient details, the prescription information including medicine type, amount, and dosage, the patient details including, name, age, pick-up location, and health insurance information;
a centralized processing system (CPS) in communication with the RCT, the health care provider uploading the prescription information and the patient details to the CPS for storage and subsequent retrieval, the CPS functioning to verify the health insurance information of the specified patient and that the medicine type is available at the identified pick-up location;
a kiosk located at the identified pick up location, the kiosk including a pharmacy vault for storing a plurality of medicine containers and a robot for handling the plurality of medicine containers, the kiosk also including system vault with a central processing unit (CPU) that is in communication with the CPS, the CPU downloading the prescription information and patient details for the specified patient, the system vault also including a user interface for allowing the identity of the specified patient to be verified;
a label generator positioned within the system vault and a labeling station accessible by the robot, the label generator functioning to create a label containing the prescription information for the particular medicine container, the labeling station functioning to apply the label to the particular medicine container to create a labeled container;
a dispenser secured within the pharmacy vault and including a secure side and a delivery side with a shelf, the dispenser being selectively rotatable between the secure and delivery sides, the robot adapted to transfer the labeled container to the shelf upon the identity of the specified patient being verified, the dispenser thereafter being rotated to the delivery side to allow the specified patient to retrieve the labeled container.

11. A system for remotely prescribing and automatically dispensing a particular medicine container to a specified patient, the system comprising:
a remote computing terminal (RCT) for use by a health care provider, the health care provider inputting into the RCT prescribing information for the specified patient as well as patient details;
a centralized processing system (CPS) in communication with the RCT, the health care provider uploading the prescription information and the patient details to the CPS for storage and subsequent retrieval;
a kiosk located at the identified pick up location, the kiosk including a pharmacy vault for storing a plurality of medicine containers and a robot for handling the plurality of medicine containers, the kiosk also including system vault with a central processing unit (CPU) that is in communication with the CPS, the CPU downloading the prescription information and patient details for the specified patient, the system vault also including a user interface for allowing the identity of the specified patient to be verified;
a dispenser secured within the pharmacy vault and including a secure side and a delivery side with a shelf, the dispenser being selectively rotatable between the secure and delivery sides, the robot adapted to transfer the labeled container to the shelf upon the identity of the specified patient being verified, the dispenser thereafter being rotated to the delivery side to allow the specified patient to retrieve the labeled container.

12. The system as described in claim 11 wherein the prescription information includes medicine type, amount, and dosage, and the patient details include name, age, pick-up location, and health insurance information.

13. The system as described in claim 11 wherein the CPS functions to verify the health insurance information of the specified patient and that the medicine type is available at the identified pick-up location.

14. The system as described in claim 11 further comprising a label generator positioned within the system vault and a labeling station accessible by the robot, the label generator functioning to create a label containing the prescription information for the particular medicine container, the labeling station functioning to apply the label to the particular medicine container to create a labeled container.

15. A kiosk for the automated dispensing of controlled substances, the kiosk comprising:
a container loading portion comprising a rotatable door on a central axis having two sides, a closed side and a shelving side;
a container dispensing portion comprising a rotatable door on a central axis having two sides, a closed portion and a shelving portion having one or more shelves; and
a container holding portion internal to said kiosk, said container holding portion having one or more shelves.

16. The kiosk of claim 15 further comprising a container labeling portion.

17. The kiosk of claim 16 further comprising a user interface.

18. The kiosk of claim 17 further comprising a central processing system.

19. The kiosk of claim 18 further comprising one or more robot systems for communicating mechanically between said container loading portion, container dispensing portion, and container holding portion by moving said one or more containers between said portions based on commands from a robot command software.

20. The kiosk of claim 19 further comprising a container labeling portion and wherein said one or more robot systems further mechanically communicates with said container labeling portion.

21. The kiosk of any of claim 20 wherein said robot command software is in communication with said user interface and said central processing system, said robot command software communicating with, and directing said robot system to manipulate containers between said container loading portion, container dispensing portion, container holding portion, and container labeling portion.

22. The kiosk of any of claim 21 further comprising one or more power supplies for providing power to said robot, said user interface, and said one or more computers running the robot command software.

23. The kiosk of any of claim 22 wherein the kiosk further comprises one or more vaults, wherein at least one vault contains the container holding portion.

24. The kiosk of claim 23 wherein said at least one vault further contains the container loading portion and said container dispensing portion, wherein said shelving side of said container loading portion and said container dispensing portion is internal to said at least one vault unless a command is received to dispense or load or dispense a controlled substance from said container loading portion or said container dispensing portion.

25. A method for remotely prescribing and automatically dispensing a medication, the method employing a remote computing terminal (RCT) that is in communication with a central processing system (CPS), the method further employing a kiosk with a CPU and a video camera, the kiosk further including a label generator and a rotatable dispenser, the method comprising the following steps:
inputting prescription information into the RCT, the prescription information identifying a particular medicine type, amount, and dosage;
inputting patient details into the RCT, the patient details including a specified patient's name and age;
uploading the prescription information and the patient details to the CPS, the CPS storing the prescription information and patient details and verifying that the specified patient is authorized to receive the particular medicine type;
the downloading prescription information and patient details to the CPU of the kiosk;
using the label generator to create a label, the label containing the prescription information and patient details, and applying the label to a container;
verifying the identity of a patient who is present at the kiosk by way of the video camera and the CPU;
moving the dispenser to allow the patient to retrieve the labeled container upon the identity of the patient being verified.

26. A device for storing and dispensing cannabis containers comprising:
a first vault with an interior area and a vault door;
a second vault with an interior area and a system access door, the system vault housing a central processing unit;
a series of storage shelves secured within the interior area of the first vault, a plurality of cannabis containers positioned upon the storage shelves;
a loading portion positioned within the first vault door, the loading portion being selectively positionable between secure and accessible orientations, with the accessible orientation allowing containers to be loaded within the first vault and the secure orientation precluding access to the first vault.

27. The device as described in claim 26 further comprising a dispensing portion secured within the first vault door, the dispensing portion being selectively positionable between secure and accessible orientations, with the accessible orientation allowing containers to be dispensed from the first vault and the secure orientation precluding access to the first vault.

28. The device as described in claim 26 wherein the loading portion rotates between the secure and accessible orientations.

29. The device as described in claim 27 wherein the dispensing portion rotates between secure and accessible orientations.

30. The device as described in claim 28 wherein the loading portion includes a shelf for supporting a cannabis container with the shelf being accessible from outside of the device when the loading portion is in the accessible orientation.

31. The device as described in claim 29 wherein the dispensing portion includes a shelf for supporting a cannabis container with the shelf being accessible from outside of the device when the dispensing portion is in the accessible orientation.

32. The device as described in claim 26 further comprising a labeling station that is accessible through a wall of the second vault, and a robot adapted to position containers within the labeling station whereby labels from the label generating device are applied.
